(19) United States
(12) Patent Application Publication
(10) Pub. No.: US 2025/0249057 A1
(43) Pub. Date: Aug. 7, 2025

(54) Synergistic Herbal Compositions for Longevity and General Health

(71) Applicant: LAILA NUTRACEUTICALS, Vijayawada, Andhra Pradesh (IN)

(72) Inventors: Ganga Raju Gokaraju, Vijayawada, Andhra Pradesh (IN); Rama Raju Gokaraju, Vijayawada, Andhra Pradesh (IN); Kiran Bhupathiraju, Vijayawada, Andhra Pradesh (IN); Venkata Kanaka Ranga Raju Gokaraju, Vijayawada, Andhra Pradesh (IN); Trimurtulu Golakoti, Vijayawada, Andhra Pradesh (IN); Venkata Krishna Raju Alluri, Vijayawada, Andhra Pradesh (IN); Venkateswarlu Somepalli, Vijayawada, Andhra Pradesh (IN); Krishanu Senguptha, Vijayawada, Andhra Pradesh (IN)

(21) Appl. No.: 18/702,917

(22) PCT Filed: Aug. 26, 2022
(86) PCT No.: PCT/IN2022/050758

§ 371 Date: Apr. 19, 2024

(30) Foreign Application Priority Data
Oct. 21, 2021 (IN) 202141047972

(51) Int. Cl. A61K 36/185; A61K 36/22; A61K 36/28; A61K 36/61; A61K 36/67; A61P 39/00

(52) U.S. Cl. CPC A61K 36/185; A61K 36/22; A61K 36/28; A61K 36/61; A61K 36/67; A61P 39/00; A61K 2236/33; A61K 2236/331; A61K 2236/51; A61K 2236/53

(57) ABSTRACT

The present invention discloses synergistic herbal compositions comprising; (i) a Punica granatum fruit extract containing metal complex of punicalagins; (ii) optionally a Punica granatum seed extract; and (iii) at least one additional ingredient selected from the extract, fraction, phytochemical or mixtures thereof derived from Mangifera indica, Piper nigrum, Tagetes erecta and Syzygium cumini; process for their preparation, methods of treatment and use of such compositions for longevity and general health.

### TECHNICAL FIELD OF THE INVENTION

The present invention relates to synergistic herbal compositions comprising; (i) a Punica granatum fruit rind extract containing metal complex or chelate or metal salt of punicalagins, punicalin, ellagic acid, and gallic acid; (ii) optionally a Punica granatum seed extract; and (iii) at least one additional ingredient selected from the extract, fraction, phytochemical or mixtures thereof derived from Mangifera indica, Piper nigrum, Tagetes erecta, and Syzygium cumini; for increasing NAD, longevity and general health.

### BACKGROUND OF THE INVENTION

Aging is characterized by the development of metabolic dysfunction and frailty. During the aging process, intracellular oxidative stress increases and the cellular nicotinamide adenine dinucleotide (NAD) pool decreases in many tissues, including the liver, skin, muscle, pancreas, and adipose tissue. Importantly, in the human brain, there is a significant decline in the NAD/NADH (NAD vs. its reduced form, NADH) redox state with age because of the gradual decline in NAD coupled with the gradual increase in NADH. Aging increases oxidative stress, which contributes to chronic low-grade inflammation. Inflammatory mediators like endotoxins and pro-inflammatory cytokines (TNF-α, IL-6) increase the expression of CD38, one of the main NADase enzymes responsible for the age-related decline of NAD. CD38 has been reported as the crucial enzyme involved in the degradation of NAD and the NAD precursor NMN in vivo, indicating CD38 has a vital role in designing strategies for increasing intercellular NAD levels and treating/age-related pathological conditions.

Nicotinamide adenine dinucleotide (NAD): Nicotinamide adenine dinucleotide, or NAD, is an endogenous biochemical molecule found in all living cells and has a key role in oxido-reductase reactions. It has been considered as a primary factor for healthy aging and longevity. Intracellular NAD level is decreased in age-related diseases, including neurodegenerative, gastrointestinal, and cardiovascular diseases. Recently, NAD boosting has been a promising therapeutic strategy for maintaining general health, retard aging, and other diseases or pathological conditions accompanied by a decline in cellular NAD levels. The ingredients that increase the cellular NAD levels or the NAD-boosters are the precursors of NAD, inhibitors of enzymes that consume NAD, or modulators of NAD synthesis pathways. Some of the NAD boosters which increased NAD levels in preclinical studies are Nicotinamide Riboside (NR), Nicotinamide mononucleotide (NMN), and CD38 inhibitors. NAD-boosters are therapeutically applicable to aging, obesity, T2DM, Skeletal muscle diseases, and myocardial diseases.

CD38 (Cluster of Differentiation 38) inhibition: CD38 is a membrane-bound protein that catalyzes the metabolism of cyclic ADP-ribose and NADP and maintains intracellular calcium. CD38 is an emerging therapeutic target under conditions in which metabolism is altered including infections, aging, and tumorigenesis. CD38 is dominantly expressed in immune cells in response to stimulation by cytokines, endotoxins, and interferons. CD38 expression causes a decline in cellular NAD levels, thus altering the availability of substrates for enzymes regulating cellular homeostasis. CD38 is identified as a critical modulator of NAD metabolism in cell signaling and aging. Recently it has been found that genetic ablation of CD38 protects against age-related NAD decline and mitochondrial dysfunction. Hence, the multi-faceted roles of CD38 provides potential opportunities to design CD38 inhibitors for various therapeutic applications. Naturally occurring flavonoids such as apigenin, quercetin, and luteolin are known to inhibit the catalytic activity of CD38. Thus, CD38 has both pathological and physiological roles and holds great promise potential as a therapeutic target in various human diseases.

Based on the above information, the inventors have postulated that an enhanced cellular NAD pool might be achieved through a combination of two fundamental approaches: up-regulating the intracellular NAD production and decreasing its breakdown through inhibition of CD38 activity. This strategy is expected to promote a positive balance towards NAD synthesis over its catabolic process and thus may help maintain a milieu of a young, healthy cell. An additional advantage of this strategy is that there will be less accumulation of unwanted metabolites in the body as compared to a high dose of NR or NMN supplementation. Besides, the CD38 inhibition strategy would also help improve immune function in aging subjects.

Hence, there is a need for safe herbal compositions for increasing NAD levels to address several age-related pathological conditions, slow down the aging process, promote healthy aging, increasing longevity, etc.

US patent U.S. Pat. No. 10,722,529B2 claims a method of modulating kynurenine to tryptophan ratio in a subject in need thereof, the method comprising administering a composition comprising a nicotinamide adenine dinucleotide (NAD) precursor to the subject, wherein the composition comprising the NAD precursor which modulates the kynurenine to tryptophan ratio in the subject. Wherein, the NAD precursor, is a nicotinamide riboside and is administered in combination with an indoleamine 2, 3-dioxygenase (IDO) antagonist.

Patent publication CN111713693A disclosed a NAD+ full nutrition formula for treating chronic diseases and resisting aging, and it is characterized by comprising the following components in parts by weight: 526-800 mg of vitamin, 291-400 mg of mineral, 15700-30000 mg of amino acid, 1300-2600 mg of extract, 840-2100 mg of fruit and vegetable powder and 3000-6000 mg of auxiliary material.

Another patent publication CN110916170A disclosed a formula for increasing in vivo NAD+ and systemic conditioning cells through total nutrition and anti-aging is characterized by comprising the following components in parts by weight: 526-800 mg of vitamin, 291-400 mg of mineral, 15700-30000 mg of amino acid, 1300-2600 mg of extract, 200-500 mg of plant enzyme, 840-2100 mg of fruit and vegetable powder and 4500-7000 mg of auxiliary material of 2000-5000 mg of probiotic powder.

US patent U.S. Pat. No. 10,106,620B2 claims a method of inhibiting the growth or proliferation of CD38-expressing cells, comprising: contacting a population of natural killer (NK) cells with an F(ab′) 2 fragment of a first anti-CD38 antibody, thereby producing a population of NK cells bound to the F(ab′) 2 fragment of the first anti-CD38 antibody; and contacting a population of CD38-expressing cells with a second anti-CD38 antibody and the population of NK cells bound to the F(ab′) 2 fragment of the first anti-CD38 antibody, thereby inhibiting growth or proliferation of the CD38-expressing cells.

Another US patent, U.S. Pat. No. 9,187,565B2, claims antibodies against CD38 for the treatment of multiple myeloma. An isolated monoclonal antibody that binds to human CD38 (SEQ ID No: 31), which does not bind to a mutant human CD38, wherein in the mutant human CD38, the serine residue in position 274 has been substituted with a phenylalanine residue (SEQ ID No: 34), and wherein the antibody inhibits the synthesis of cGDPR by at least 25% after 90 minutes at a concentration of 3 μg/ml.

Therefore, there remains a need in the art for safe and efficient herbal compositions for delaying aging process, promoting anti-aging activity, promoting longevity, improving general health; delaying/preventing/treating neurodegenerative, cardiovascular, and gastrointestinal health diseases/conditions; improving glucose tolerance, muscle function, exercise capacity and cardiac function, neuro-behavioral functions including cognition and locomotor activity, endothelial function and mitochondrial function; and reducing oxidative stress, etc., by boosting NAD levels in humans.

### OBJECT OF THE INVENTION

Therefore, the primary object of the present invention is to provide synergistic and safe herbal compositions comprising; (i) a Punica granatum fruit rind extract containing a metal complex or chelate or metal salt of at least one phytochemical selected from punicalagins, punicalin, ellagic acid, and gallic acid or mixtures thereof; wherein the metal is selected from magnesium, calcium, and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; for longevity and general health.

Another object of the present invention is to provide synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) further containing at least one optional component selected from pharmaceutically or nutraceutically or dietically acceptable excipients, carriers, and diluents.

Another object of the present invention is to provide a process for the preparation of synergistic herbal compositions comprising; (i) a Punica granatum fruit rind extract containing a metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; (v) further at least one optional component selected from pharmaceutically or nutraceutically or dietically acceptable excipients, carriers, and diluents.

Yet, another object of the invention is to provide a method of obtaining at least one health benefit related to amelioration of the conditions associated with aging or age-related decline in NAD levels in human subject, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress; wherein the method comprises supplementing the said human subject with an effective dose of a composition comprising; (i) a Punica granatum fruit rind extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) further at least one optional component selected from pharmaceutically or nutraceutically or dietically acceptable excipients, carriers, and diluents.

Yet, another object of the invention is to provide use of the present herbal composition comprising; (i) a Punica granatum fruit rind extract containing metal complex or chelate or metal salt of at least one phytochemical selected from punicalagins, punicalin, ellagic acid and gallic acid or mixtures thereof; wherein the metal is selected from magnesium, calcium and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini, for obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

### SUMMARY OF THE INVENTION

The present invention provides synergistic herbal compositions comprising; (i) a Punica granatum fruit rind extract containing metal complex or chelate or metal salt of at least one phytochemical selected from punicalagins, punicalin, ellagic acid and gallic acid or mixtures thereof; wherein the metal is selected from magnesium, calcium and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) further containing optionally at least one component selected from pharmaceutically or nutraceutically or dietically acceptable excipients, carriers, and diluents; for obtaining health benefits related to amelioration of the conditions associated with aging or age-related decline in NAD levels, which are selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

Another aspect of the present invention is to provide synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; (v) optionally further containing at least one component selected from pharmaceutically or nutraceutically or dietically acceptable excipients, carriers, and diluents.

Another aspect of the present invention provides method of obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress; wherein the method comprises supplementing human with an effective dose of compositions comprising; (i) a Punica granatum fruit rind extract containing metal complex or chelate or metal salt of at least one phytochemical selected from punicalagins, punicalin, ellagic acid and gallic acid or mixtures thereof; wherein the metal is selected from magnesium, calcium and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) optionally further containing at least one component selected from pharmaceutically or nutraceutically or dietically acceptable excipients, carriers, and diluents.

A further aspect of the present invention provides use of herbal compositions comprising; (i) a Punica granatum fruit rind extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) optionally further at least one component selected from pharmaceutically or nutraceutically or dietically acceptable excipients, carriers, and diluents for obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

In another aspect, the present invention provides a process for the preparation of compositions comprising; (i) a Punica granatum fruit rind extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; (v) optionally further at least one component selected from pharmaceutically or nutraceutically or dietically acceptable excipients, carriers, and diluents.

### DETAILED DESCRIPTION OF THE INVENTION

The invention will now be described in detail in connection with certain preferred and optional embodiments so that various aspects thereof may be more fully understood and appreciated.

The terms “metal complex,” “metal chelate” and “metal salt” convey the same meaning and are used interchangeably in the specification. Thus, Punica granatum fruit rind extract contains metal complex of at least one phytochemical selected from punicalagins, punicalin, ellagic acid and gallic acid; Punica granatum fruit rind extract contains a metal chelate of at least one phytochemical selected from punicalagins, punicalin, ellagic acid, and gallic acid; and Punica granatum fruit rind extract contains metal salt of at least one phytochemical selected from punicalagins, punicalin, ellagic acid, and gallic acid; convey the same meaning and are interchangeably used throughout in the specification. The terms “improve,” and “better,” as used herein, convey the same meaning and are interchangeable. The terms “fruit peel,” “fruit rind” as used herein, conveys the same meaning and is interchangeable. Unless specified otherwise, all technical and scientific terms used herein have the same meaning as commonly understood by an ordinary skill in the art to which this invention belongs. To describe the invention, certain terms are defined herein specifically as follows.

The source of the herbs used in the invention is as follows:
1. Punica granatum was collected from Potulanage Palli village, Dharmavaram Mandal, Ananthapur district, Andhra Pradesh.
2. Mangifera indica was collected from Gudavalli village, Vijayawada rural, Krishna district, Andhra Pradesh.
3. Piper nigrum was collected from Aswaraopet village, Bhadradri Kothagudem district, Telangana.
4. Tagetes erecta was collected from Rayapadu village, Tallur Mandal, Guntur district, Andhra Pradesh.
5. Syzygium cumini was collected from Kankipadu village, Kankipadu Mandal, Krishna district, Andhra Pradesh.

Nicotinamide adenine dinucleotide (NAD) is an essential cofactor of dehydrogenase and its metabolism is a dynamic redox cycle that functions to shuttle electrons throughout cells to maintain redox homeostasis and bioenergetics. Playing a vital role in energy metabolism in eukaryotic cells, NAD+ accepts hydride equivalents, to form reduced NADH, which furnishes reducing equivalents to the mitochondrial electron transport chain (ETC) to fuel oxidative phosphorylation. NAD+ is synthesized in cells through three different pathways, (i) de novo synthesis from tryptophan which accounts for minority of the total NAD+ pool; (ii) from nicotinic acid via the Preiss-Handler pathway; and (iii) from recycling nicotinamide (NAM), a NAD+ breakdown product via Salvage pathway through NAM phosphoribosyl transferase (NAMPT) to nicotinamide mononucleotide (NMN) and back to NAD+ via NMN adenyltransferases 1-3 (NMNATs).

A tight balance between NAD biosynthesis and its utilization machinery controls the cellular NAD pool in a healthy cell. During the aging process and in intracellular stress, the cellular NAD pool decreases in many tissues, including the liver, skin, muscle, pancreas, and adipose tissue. Aging increases oxidative stress, which contributes to chronic low-grade inflammation. Inflammatory mediators like endotoxins and proinflammatory cytokines (TNF-α, IL-6) increase the expression of CD38, one of the main NADase enzymes responsible for the age-related decline of NAD. It is a multifunctional protein which plays important roles in several physiological processes such as, nuclear Ca²⁺ homeostasis, immunity, inflammation, as well as glucose and lipid homeostasis. CD38 has been reported as the crucial enzyme involved in the degradation of the NAD and its precursor NMN in vivo, indicating that CD38 has a key role in the modulation of the intracellular NAD pool. A reciprocal relationship of increasing CD38 activity with decreasing NAD levels exists in cells in parallel with declining mitochondrial function as aging progresses.

Based on the above information, we hypothesize that an enhanced cellular NAD pool can be achieved through a combination of two fundamental approaches: up-regulating the intracellular NAD production and decreasing its breakdown through inhibition of CD38 activity. This strategy is expected to synergistically promote a positive balance towards NAD synthesis over its catabolic process and thus may help maintain a milieu of a young, healthy cell. Hence, to provide safe herbal compositions to address the problems associated with age-related decline in NAD levels, the inventors strategized to screen a large number of plant extracts to evaluate their efficacies in increasing intracellular NAD and inhibiting CD38 (NADase) activity and then combine the selected extracts to prepare synergistic combinations, which may provide a net benefit of increasing intracellular NAD levels.

The inventors of the current application randomly screened a large number of plant extracts and their fractions to increase cellular NAD levels and inhibit CD38 enzyme activity. They found that the Punica granatum fruit rind extract containing metal complex, Punica granatum seed extract, Mangifera indica seed extract, Tagetes erecta (marigold) flower extract, Piper nigrum seed extract, and Syzygium cumini fruit extract show good efficacies in naturally enhancing NAD synthesis and inhibiting CD38 activity.

A brief summary of each of the plant materials used in the preferred embodiment of the invention is provided herein below.

Punica granatum fruit: P. granatum (pomegranate) is cultivated and consumed globally. It is one of the important fruit with a broad spectrum of health benefits for humans and has been consumed for centuries for the prevention and treatment of a wide range of health issues. The rind contains punicalagins, punicalins, ellagic acid, and gallic acid. Punicalagins, the major chemical constituents of P. granatum rind extract, are unique to pomegranate and they are part of a family of ellagitannins (polyphenols). Punicalagins means a mixture of punicalagin A (punicalagin α) and punicalagin B (punicalagin β), as shown in the FIGURE below, and hence the term “Punicalagins” as referred to in the entire specification has the same meaning.

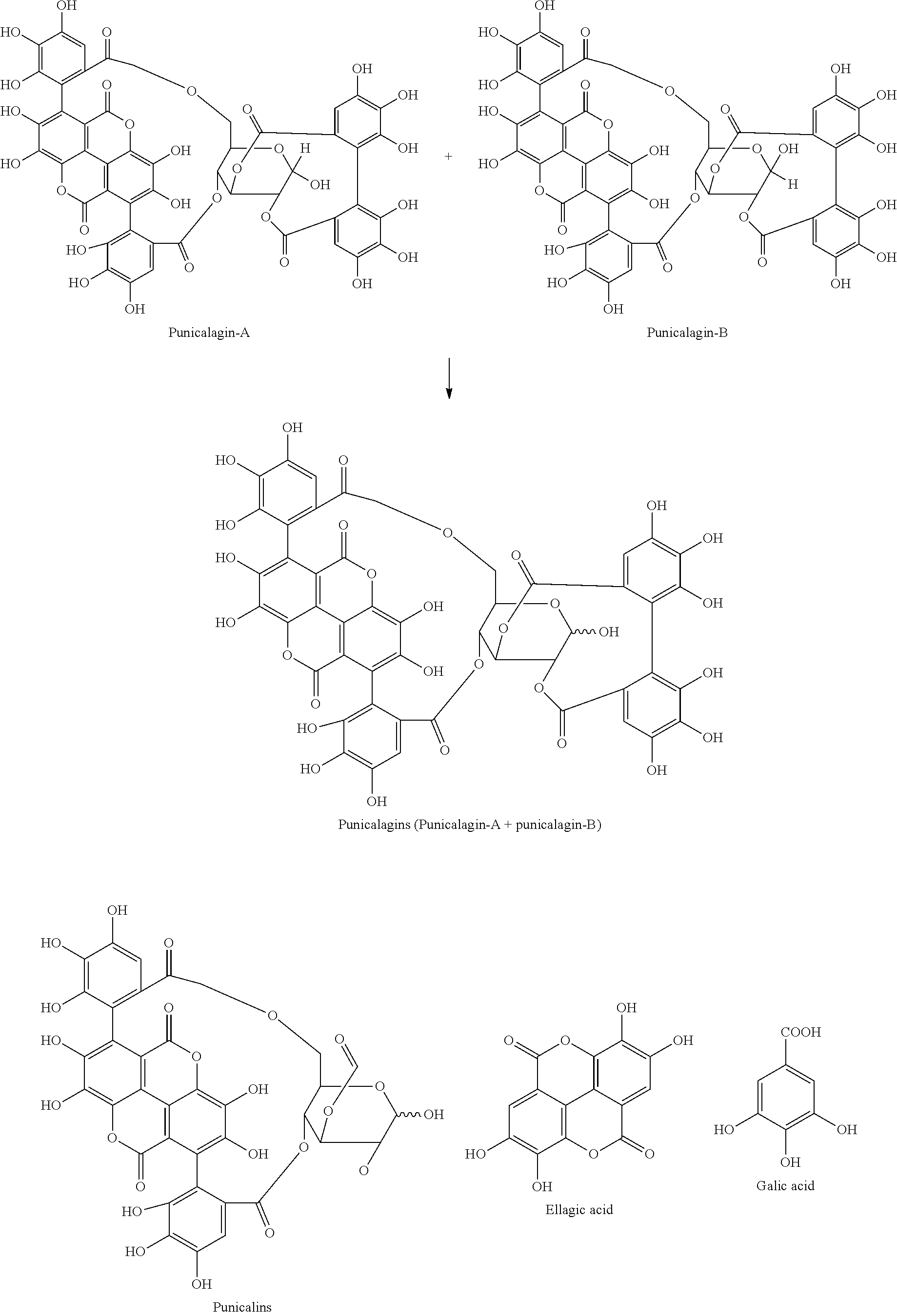

FIG. 1: Chemical structures of punicalagins, punicalins, ellagic acid, and gallic acid.

Punica granatum seed: P. granatum seeds are edible and are a rich source of dietary fiber. Punicic acid (9Z,11E,13Z-octadeca-9,11,13-trienoic acid) is the major chemical constituent of seeds which exists as its triglyceride, 1,2,3-tripunicyl glycerol, and is also referred as “tripunicyl glycerol” in the entire specification. Thus “1,2,3-tripunicyl glycerol”, “tripunicyl glycerol” “tripunicic acid ester” and “punicic acid” conveys the same meaning and are used interchangeably in the specification.

Mangifera indica seed: M. indica, commonly known as mango and aam, belongs to the genus Mangifera of the family Anacardiaceae in kingdom-Planate. M. indica seed consists of a tenacious coat enclosing the kernel. The chemical constituents in seeds include polyphenols such as quercetin, kaempferol, gallic acid, tannin, and xanthone, and the seeds are reported to have various pharmacological activities. Pentagalloyl glucose (1,2,3,4,6-pentagalloylglucose) is isolated as one of the major gallotannin in M. indica seed.

Piper nigrum seed: P. nigrum (black pepper), a common spice and seasoning around the globe, is a member of Piperaceae family, and has several uses. The chemical constituents in the fruit include lignans derivatives, phenolics, terpenes, chalcones, alkaloids, steroids, flavonoid, dihydropipericide, N-trans-feruloyltryamine, and isobutyllocadienamide. P. nigrum and its major component, piperine, have several pharmacological activities.

Tagetes erecta L: Marigold is a large flowered annual herbaceous plant belonging to Asteraceae, and different parts of this plant, including flowers, are used in folk medicine to cure various diseases. Marigold flowers are one of the most popular edible flowers worldwide and are used in salads and as natural food colorants. Marigold flowers are a rich source of lutein, lutein esters, zeaxanthin, polyphenols, and flavonoids. Among flavonoids, quercetagetin is the major chemical constituent responsible for many biological activities.

For the first time, the inventors surprisingly found that the metal complex of Punica granatum (pomegranate) extract, especially punicalagins, has better NAD boosting and CD38 inhibition activities compared to the Punica granatum extract, where the metal is absent.

Metal ions are inorganic substances required by the body in small amounts for a variety of biological functions. Magnesium is a nutrient that the body needs to stay healthy. It is one of the six essential metal ions for the human body, with a requirement of around 400 mg/day. Magnesium is important for many biological processes in the body, including regulating muscle and nerve function, blood sugar levels, blood pressure, bone health, calcium absorption, relieving anxiety, etc. Similarly, potassium, and calcium are also required by the body for various biological functions.

Punica granatum fruit rind extract containing a metal complex of punicalagins: The pomegranate fruit rind water extract was prepared and treated with magnesium oxide to form a magnesium complex of punicalagins, punicalin, ellagic acid, and gallic acid (PGRMg-1). The extract thus obtained contains 9.38% of punicalagins, 2.13% of punicalin, 2.32% of ellagic acid and 0.50% of gallic acid, and 0.76% of magnesium. Similarly, pomegranate fruit rind 70% aqueous ethanol extract containing magnesium complex was also prepared (examples 1 & 2). For comparison, pomegranate fruit rind water regular extract without complex (PGR) was also prepared, which contain 9.20% of punicalagins. Further, the whole pomegranate fruit (contains fruit rind and seed) water extract containing magnesium complex of punicalagins was also prepared (example 6).

Complex formation: P. granatum fruit rind water extract contains punicalagins, punicalin, ellagic acid, and gallic acid as the major phytochemical along with other phytochemicals. Punicalagins, punicalin and ellagic acid contain several hydroxyl groups (FIG. 1) capable of forming complexes with metals. Further gallic acid has additionally carboxylic acid group capable of forming complexes with metals. Magnesium complex of P. granatum fruit rind water extract containing punicalagins and other phenolic compounds was prepared by using magnesium oxide. The process was typically performed in water, followed by fine filtration to remove any unreacted magnesium oxide. ICP-MS analysis of the resulting product showed the presence of magnesium (0.76%). As magnesium oxide is practically insoluble in water, the only possibility for the existence of magnesium in the product is by forming a complex with punicalagins or other phenolic compounds.

Further, P. granatum fruit rind water extract containing punicalagins, punicalin, ellagic acid, and gallic acid and magnesium (PGRMg-1) showed pH of about 5.1, whereas P. granatum fruit rind water extract without magnesium (PGR) showed pH of about 3.4. The difference in pH value clearly suggests the formation of magnesium complex with punicalagins and/or other phenolic compounds and can be differentiated with P. granatum fruit rind water extract without magnesium (PGR).

The Punica granatum fruit rind extracts with or without metal are screened for their NAD boosting and CD38 inhibition activities, and the results are presented in Table 1. Unexpectedly, the P. granatum fruit rind extract containing magnesium complex of punicalagins (example 1) showed better efficacy in increasing NAD levels and inhibiting CD38 when compared to the corresponding extract without metal (Comparative example 1A).

TABLE 1
NAD boosting and CD38 inhibition activities of
P. granatum fruit rind extract containing
punicalagins and magnesium and regular extract

| | | % increase of | % inhibition of |
|---|---|---|---|
| | | NAD over | CD38 over |
| Example | | control at 1 | control at 10 |
| # | Extract | (μg/mL) | (μg/mL) |
| 1A | P. granatum fruit rind | 15.30 | 19.92 |
| | water regular extract | | |
| | (PGR) | | |
| 1 | P. granatum fruit rind | 28.49 | 32.19 |
| | water extract containing | | |
| | punicalagins and | | |
| | magnesium (PGRMg-1) | | |

Thus, NAD-boosting activity of P. granatum fruit rind water extract containing magnesium complex of punicalagins (PGRMg-1) showed a 28.49% increase in NAD over control at 1 μg/mL. Whereas P. granatum fruit rind water regular extract (PGR) showed only 15.30% increase in NAD over control at 1 μg/mL, which is a surprising and unexpected result for enhancement of NAD activity of the P. granatum fruit rind extract containing magnesium complex of punicalagins (PGRMg-1) compared to the corresponding P. granatum fruit rind water regular extract (PGR) (Table-1). Similarly, CD38 inhibition activity of P. granatum fruit rind water extract containing magnesium complex of punicalagins also showed better efficacy in inhibition of CD38 activity (Table-1), which a surprising and unexpected result.

Hence, the inventive Punica granatum fruit rind extract containing metal complex of punicalagins, wherein the punicalagins are in the range of 1-40%, and metal is in the range of 0.1-5.0%, surprisingly showed a better efficacy in NAD boosting and CD38 inhibition activities compared to the regular extract without a metal salt.

### Compositions

Encouraged by the increased efficacy in NAD boosting and CD38 inhibition activities of Punica granatum fruit rind extract containing metal complex of punicalagins, punicalin, ellagic acid, and gallic acid; the inventors prepared several compositions comprising, a Punica granatum fruit rind extract containing metal complex of at least one phytochemical selected from punicalagins, punicalin, ellagic acid and gallic acid or mixtures thereof; wherein the metal is selected from magnesium, calcium and potassium; and optionally containing Punica granatum seed extract; and in combination with at least one ingredient selected from the extracts, fractions, phytochemicals or mixtures thereof derived from Mangifera indica, Tagetes erecta, Piper nigrum and Syzygium cumini.

The inventors have also prepared several compositions comprising, a Punica granatum fruit rind extract containing metal complex or chelate or salt of at least one phytochemical selected from punicalagins, punicalin, ellagic acid and gallic acid or mixtures thereof; and Punica granatum seed extract.

Further, the inventors have also prepared several compositions comprising, a Punica granatum whole fruit extract containing metal complex or chelate or salt of at least one phytochemical selected from punicalagins, punicalin, ellagic acid and gallic acid or mixtures thereof; and in combination with at least one ingredient selected from the extracts, fractions, phytochemicals or mixtures thereof derived from Mangifera indica, Tagetes erecta, Piper nigrum and Syzygium cumini.

Thus, various solvent extracts of Punica granatum seed, Mangifera indica seed, Tagetes erecta flower, Piper nigrum fruit and Syzygium cumini fruit were also prepared with different solvents.

Punica granatum seed extracts: Seeds were pulverized, and the powder was extracted with various solvents such as ethyl acetate, water followed by 50% aqueous ethanol, and ethyl acetate followed by 50% aqueous ethanol and water; to obtain P. granatum seed ethyl acetate extract (PGS-1), P. granatum seed extract blend (PGS-2) of water extract and 50% ethanol extract obtained sequentially, and P. granatum seed extract blend (PGS-3) of ethyl acetate, 50% aq. ethanol extract and water extract obtained sequentially. These extracts were standardized to tripunicyl glycerol or punicic acid by analytical HPLC method and are summarized in example-5.

Mangifera indica, Tagetes erecta (Marigold), Piper nigrum and Syzygium cumini extracts: Mangifera indica seed kernel, Tagetes erecta (Marigold) flower, Piper nigrum fruit and Syzygium cumini fruit were pulverized and the powders were extracted separately with various solvents to obtain the corresponding extracts, and the extracts were standardized with their corresponding phytochemical markers using analytical HPLC method. The results are summarized in examples 7-10.

For illustration, four compositions (comp #2, 2A, 8 and 8A) containing (a) Punica granatum fruit rind water extract containing magnesium complex of punicalagins, punicalin, ellagic acid, and gallic acid (PGRMg-1), Punica granatum seed extract (PGS-2), and Mangifera indica seed extract (MI-4) in the ratio of 6:2:2 and (b) P. granatum fruit rind water regular extract (PGR), P. granatum seed extract (PGS-2) and M. indica seed extract (MI-4) in the ratio of 6:2:2, a comparative composition without magnesium complex; (c) Punica granatum fruit rind water extract containing magnesium complex of punicalagins, punicalin, ellagic acid, and gallic acid (PGRMg-1), Punica granatum seed extract (PGS-2) and Piper nigrum fruit extract (PN-4) in the ratio of 6:2:2 and (d) P. granatum fruit rind water regular extract (PGR), P. granatum seed extract (PGS-2) and P. nigrum fruit extract (PN-4) in the ratio of 6:2:2, a comparative composition without magnesium complex; were prepared. These compositions were tested for their efficacy in boosting NAD levels and inhibiting CD38 in vitro cellular models, and the results are summarized in Table 2.

TABLE 2
Efficacy of compositions comprising Punica granatum fruit rind extract with or without
magnesium and Piper nigrum fruit extract or Mangifera indica seed extract

| | | | % |
|---|---|---|---|
| | | % increase | inhibition |
| | | of NAD at | of CD38 at |
| Comp # | Composition description | 1 μg/mL | 10 μg/mL |
| Comp-2A | P. granatum fruit rind water extract | 29.87 | 31.57 |
| | (PGR), P. granatum seed extract | | |
| | (PGS-2), and M. indica seed extract | | |
| | (MI-4) in the ratio of 6:2:2. | | |
| Comp-2 | P. granatum fruit rind water extract | 41.50 | 48.77 |
| | containing magnesium (PGRMg-1), P. | | |
| | granatum seed extract (PGS-2), and M. | | |
| | indica seed extract (MI-4) in the ratio | | |
| | of 6:2:2. | | |
| Comp-8A | P. granatum fruit rind water extract | 27.73 | 29.24 |
| | (PGR), P. granatum seed extract | | |
| | (PGS-2), and P. nigrum fruit extract | | |
| | (PN-4) in the ratio of 6:2:2 | | |
| Comp-8 | P. granatum fruit rind extract | 38.96 | 42.89 |
| | containing magnesium (PGRMg-1), P. | | |
| | granatum seed extract (PGS-2), and P. | | |
| | nigrum fruit extract (PN-4) in the ratio | | |
| | of 6:2:2 | | |

From the table-2, the composition-2 comprising P. granatum fruit rind water extract containing magnesium complex of punicalagins, punicalin, ellagic acid, and gallic acid (PGRMg-1), P. granatum seed extract (PGS-2), and M. indica seed extract (MI-4) in the ratio of 6:2:2 showed 41.50% increase in NAD level at 1 μg/mL. While the similar composition (Composition 2A) with P. granatum fruit rind water extract without magnesium complex (PGR) in the same ratio of the other ingredients showed only 29.87% increase at the same concentration (1 μg/mL). This increase in cellular NAD levels of the inventive composition (composition-2) is a surprising and unexpected result. Similarly, the composition-2 comprising magnesium complex of punicalagins, punicalin, ellagic acid, and gallic acid (PGRMg-1) showed significantly better inhibition of CD38 activity than the similar composition without magnesium complex; this is also a surprising and unexpected result. Similarly, composition-8 comprises P. granatum fruit rind extract containing magnesium (PGRMg-1), P. granatum seed extract (PGS-2), and P. nigrum fruit extract (PN-4) in the ratio of 6:2:2; also showed surprisingly higher efficacy in increasing NAD and inhibiting CD38 activity than the corresponding composition without magnesium complex (comp-8A).

Similarly, four other compositions (comp #25, 25A, 32 and 32A) containing (e) Punica granatum fruit rind water extract containing magnesium complex of punicalagins, punicalin, ellagic acid, and gallic acid (PGRMg-1) and Tagetes erecta (marigold) flower extract (TE-2) in the ratio of 3:1 and (f) P. granatum fruit rind water regular extract (PGR), and marigold extract (TE-2) in the ratio of 3:1 (a comparative composition without magnesium complex); and (g) Punica granatum fruit rind water extract containing magnesium complex of punicalagins, punicalin, ellagic acid, and gallic acid (PGRMg-1) and Syzygium cumini fruit extract (SC-2) in the ratio of 1:1; and (h) P. granatum fruit rind water regular extract (PGR), and S. cumini fruit extract (SC-2) in the ratio of 1:1, (also a comparative composition without magnesium complex); were prepared. These compositions were tested for their efficacy in boosting NAD levels and inhibiting CD38 in vitro cellular models, and the results are summarized in Table 3.

TABLE 3
Efficacy of compositions comprising Punica granatum fruit rind extract with or without
magnesium and Tagetes erecta flower extract or Syzygium cumini fruit extract

| | | | % |
|---|---|---|---|
| | | % increase | inhibition |
| | | of NAD at | of CD38 at |
| Comp # | Composition description | 1 μg/mL | 10 μg/mL |
| Comp- | P. granatum fruit rind water extract | 31.29 | 35.68 |
| 25A | (PGR), and marigold flower extract | | |
| | (TE-2) in the ratio of 3:1. | | |
| Comp-25 | P. granatum fruit rind water extract | 44.56 | 53.45 |
| | containing magnesium (PGRMg-1) | | |
| | and marigold flower extract (TE-2) in | | |
| | the ratio of 3:1. | | |
| Comp- | P. granatum fruit rind water extract | 29.43 | 33.87 |
| 32A | (PGR), S. cumini fruit extract (SC-2) | | |
| | in the ratio of 1:1 | | |
| Comp-32 | P. granatum fruit rind extract | 40.19 | 46.13 |
| | containing magnesium (PGRMg-1) | | |
| | and S. cumini fruit extract (SC-2) in | | |
| | the ratio of 1:1 | | |

From the table-3, the NAD-boosting activity of the composition-25 comprising P. granatum fruit rind water extract containing magnesium complex of punicalagins, punicalin, ellagic acid, and gallic acid (PGRMg-1) and marigold extract (TE-2) in the ratio of 3:1 showed a 44.56% increase cellular NAD level at 1 μg/mL. While the similar composition-25A of P. granatum fruit rind water extract without magnesium (PGR) in the same ratio of 3:1 showed a 31.29% increase at 1 μg/mL, which is 13.27% lower than the similar composition containing the magnesium complex. This increased NAD level of the inventive composition is a surprising and unexpected result. Further, the composition-25 also showed significantly better inhibition of CD38 activity than the equivalent composition without magnesium (Comp-25A); this is also a surprising and unexpected result. Similarly, composition-32 comprising P. granatum fruit rind extract containing magnesium (PGRMg-1) and S. cumini fruit extract (SC-2) in the ratio of 1:1; also showed surprisingly better efficacy both in increasing NAD and inhibiting CD38 activity, when compared to the composition without the magnesium (comp-32A).

Hence, the compositions of Punica granatum fruit rind extract containing magnesium complex of punicalagins, punicalin, ellagic acid, and gallic acid (PGRMg-1) with other herbal extracts showed better efficacy in boosting NAD levels and inhibiting CD38 when compared to the corresponding compositions without magnesium.

Subsequently, various compositions (C1-C24) of (i) Punica granatum fruit rind extract containing metal complex or chelate of at least one phytochemical selected from punicalagins, punicalin, ellagic acid, and gallic acid or mixtures thereof; (ii) Punica granatum seed extract; (iii) one additional ingredient selected from the extracts derived from Mangifera indica, and Piper nigrum were prepared as described in examples 11-14.

Similarly, various compositions (C25-C27) of (i) Punica granatum fruit rind extract containing metal complex or chelate of at least one phytochemical selected from punicalagins, punicalin, ellagic acid, and gallic acid or mixtures thereof; (ii) one additional ingredient selected from the extracts derived from Mangifera indica, Tagetes erecta (marigold), Syzygium cumini and Piper nigrum were prepared as described in examples 15-17.

Further, various compositions (C38-C40) containing Punica granatum whole fruit extract, comprising of (i) Punica granatum whole fruit extract contains metal complex or chelate of at least one phytochemical selected from punicalagins, punicalin, ellagic acid, and gallic acid or mixtures thereof; (ii) one additional ingredient selected from the extracts derived from Mangifera indica, Tagetes erecta (marigold), Syzygium cumini and Piper nigrum were prepared as described in example 18. Furthermore, various compositions (C41-43) of (i) Punica granatum fruit rind extract containing metal complex; and (ii) Punica granatum seed extract were also prepared as described in example 19.

Then, these compositions (comp 1-43) were tested for their efficacy in boosting NAD and inhibition of CD38 in vitro cellular models compared to the corresponding individual ingredients. Unexpectedly, these compositions showed synergistic activity in boosting NAD and inhibiting CD38 levels.

For example, Punica granatum fruit rind water extract containing magnesium complex of punicalagins, punicalin, ellagic acid, and gallic acid (PGRMg-1) at 0.8 μg/mL concentration, P. granatum seed extract (PGS-2) at 0.1 μg/mL concentration and Mangifera indica seed extract (MI-4) at 0.1 μg/mL concentration showed 22.79%, 2.29% and 2.61% increase in NAD levels respectively over control. The composition-1 (C-1) containing PGRMg-1, PGS-2, and MI-4 in the ratio of 8:1:1 at 1 μg/mL showed a 36.87% increase in NAD levels, which is significantly higher than the additive effect of 27.69% (22.79+2.29+2.61) calculated from the increase in NAD levels shown by the corresponding individual ingredients. The compositions 2-6 (C-2 to C-6) containing these three extracts at other ratios also exhibited synergism compared to the increase of NAD levels shown by each of their corresponding individual ingredients, as summarized in Table 4. Similarly, these compositions also showed synergistic CD38 inhibitions, when compared to the corresponding individual ingredients as summarized Table 13.

Similarly, the compositions containing various solvent extracts of Punica granatum fruit rind extracts containing magnesium, Punica granatum seed extracts, and Mangifera indica, or Piper nigrum also showed synergistic efficacy in increasing NAD levels and inhibiting CD38 activity compared to the corresponding individual ingredients (Tables 5-7 and Tables 14-16 respectively).

In another example, the compositions containing Punica granatum fruit rind extracts containing magnesium complex of punicalagins, punicalin, ellagic acid, and gallic acid and Tagetes erecta (marigold) extracts or Syzygium cumini extracts also showed synergistic efficacy in increasing NAD levels (Tables 8-10) and inhibiting CD38 activity (Tables 17-19) than the corresponding individual ingredients.

In another example, the compositions containing Punica granatum whole fruit extract containing magnesium complex of punicalagins, punicalin, ellagic acid, and gallic acid and Mangifera indica seed kernel extract also showed synergistic efficacy in increasing NAD levels (Table 11) and inhibiting CD38 activity (Table 20) compared to their corresponding individual ingredients.

In another example, the compositions containing Punica granatum fruit rind extract containing the magnesium complex of punicalagins, punicalin, ellagic acid, and gallic acid and Punica granatum seed extract also showed synergistic efficacy of increased NAD levels (Table 12) and CD38 inhibitions (Table 21) compared to their corresponding individual ingredients.

Thus, the compositions comprising; (i) Punica granatum fruit rind extract containing metal complex or chelate or metal salt of at least one phytochemical selected from punicalagins, punicalin, ellagic acid, and gallic acid or mixtures thereof; wherein the metal is selected from magnesium, calcium, and potassium; (ii) optionally Punica granatum seed extract; (iii) at least one additional ingredient selected from the extract, fraction, phytochemical or mixtures thereof derived from Mangifera indica, Piper nigrum, Tagetes erecta, and Syzygium cumini; have the unexpected tendency to show synergistic efficacy in increasing NAD levels and inhibiting CD38 activity when compared to their individual ingredients.

Process: The process for the preparation of compositions comprising (i) a Punica granatum fruit rind extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; which consists of the following steps;
(a) extracting dried Punica granatum fruit powder with a suitable solvent;
(b) treating the solution with a metal compound;
(c) filtering the solution;
(d) evaporating the solvent and drying the residue to obtain P. granatum fruit extract containing metal complex;
(e) optionally adding Punica granatum seed extract;
(f) blending with at least one extract derived from Mangifera indica, Tagetes erecta, Piper nigrum, and Syzygium cumini; in the presence of a suitable solvent; and optionally suitable excipients;
(g) drying the product to get the composition.

The suitable solvent used in the process for the preparation of compositions is selected from but not limited to C1-C5 alcohols, like ethanol, methanol, propanol, n-butanol, ethyl acetate, acetone, water, and mixtures thereof.

The metal used in the process for the preparation of the compositions is selected from magnesium, calcium, and potassium, and the metal compound used for the preparation of these compositions is in the form of their metal salts, metal oxides, metal hydroxides, or carbonates. Examples include magnesium oxide, magnesium carbonate, magnesium hydroxide, calcium oxide, calcium hydroxide, calcium carbonate, potassium hydroxide, and potassium carbonate.

Formulations: The synergistic herbal compositions comprising; (i) a Punica granatum fruit extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; can be formulated with at least one component selected from pharmaceutically or nutraceutically or dietically acceptable excipients, carriers and diluents.

The synergistic compositions can be formulated using a pharmaceutically or nutraceutically or dietically acceptable excipients, carriers and diluents; selected from monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as starch and modified starch such as sodium starch glycolate, pre-gelatinized starch, soluble starch, ultrasperse A and other modified starches; Dextrins that are produced by hydrolysis of starch or glycogen such as yellow dextrin, white dextrin, maltodextrin, glucidex 12D, rice maltodextrin etc.; Polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide, syloids etc.; metallic stearates such as but not limited to calcium stearate, magnesium stearate, zinc stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; Natural pigments, flavors, Class I & Class II preservatives and aqueous, alcoholic, hydro-alcoholic, organic solutions of above listed ingredients alone or in combination.

The forgoing demonstrates that herbal compositions comprising; (i) a Punica granatum fruit rind extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta (marigold) and Syzygium cumini; show better efficacy in boosting the production of NAD and inhibition of CD38 activities when compared to their corresponding individual ingredients. Hence, this is unexpected synergistic activity for the said compositions and as such they can be useful for improving/ameliorating conditions associated with aging and age-related decline in NAD levels.

### Increasing NAD Levels in Aged Animals:

Recent preclinical and clinical studies have demonstrated that supplementation of NAD precursors (such as NMN, NR, and nicotinamide) or compounds that inhibits the activity of the NAD-degrading enzyme (CD38, or PARPs) can increase the intracellular NAD pool in tissues, thereby alleviating aging symptoms and extending healthy life. Therefore, an increase in the intracellular pool of NAD levels has been established as a potential strategy to fight the progression of aging symptoms or promote healthy aging or delay the aging process.

As demonstrated earlier, the herbal ingredients and their combinations increased cellular NAD levels and inhibited CD38 enzyme activity in cellular models in vitro. To check if this in vitro activity can be translated into an animal model, we have conducted an in vivo proof-of-concept study. We evaluated the efficacy of the selected herbal extracts and their combinations in increasing the NAD levels in the aged Wistar rats.

Group-wise summary of blood NAD concentrations in the aged animals supplemented with extracts and their compositions was presented in example 23 and table-22. The values are present as mean NAD concentrations in the blood samples. A mean NAD concentration in the blood samples of the young animals is also presented in parallel for comparison. The table also shows the mean increase in NAD levels in the respective group of animals on day 60 from day 1.

The present compositions showed synergistic efficacy in increasing NAD levels in the experimental animals. For example, Punica granatum fruit rind water extract containing magnesium complex of punicalagins, punicalin, ellagic acid, and gallic acid (PGRMg-1), P. granatum seed extract (PGS-2), and Mangifera indica seed extract (MI-4) showed 16.82%, 9.72%, and 13.85% increase respectively in NAD levels, when compared to their corresponding levels on day 1 (base line). The composition-44 containing these three extracts at 6:2:2 ratio along with excipients showed a 27.84% increase in NAD levels (Table 22), which is a significantly higher increase than the efficacy shown by the corresponding individual ingredients, suggesting an in vivo synergistic effect among these three ingredients. Similarly, Punica granatum fruit rind water extract containing magnesium complex of punicalagins, punicalin, ellagic acid, and gallic acid (PGRMg-1) and marigold extract (TE-2) showed 16.82% and 15.29% increase in NAD levels, respectively, when compared to their base line levels (day 1) (Table 22). The composition-47 containing these two extracts at 3:1 ratio along with excipients showed 31.03% increase in NAD levels, which is also a significantly higher increase than the corresponding individual ingredients, suggesting a synergistic effect between these two extracts in increasing NAD levels.

Therefore, in an important embodiment, the present invention provides synergistic herbal compositions comprising; (i) a Punica granatum fruit rind extract containing metal complex or chelate or metal salt of at least one phytochemical selected from punicalagins, punicalin, ellagic acid and gallic acid or mixtures thereof; wherein the metal is selected from magnesium, calcium and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta (marigold) and Syzygium cumini; (iv) further at least one optional component selected from pharmaceutically or nutraceutically or dietically acceptable excipients, carriers, and diluents; for obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

In one preferred embodiment, the present invention provides synergistic herbal compositions comprising; (i) a Punica granatum fruit peel/rind extract containing magnesium complex or chelate or metal salt of at least one phytochemical selected from punicalagins, punicalin, ellagic acid and gallic acid or mixtures thereof; (ii) a Punica granatum seed extract; and (iii) at least one additional ingredient selected from the extract, fraction, phytochemical or mixtures thereof derived from Mangifera indica, Piper nigrum, Tagetes erecta, and Syzygium cumini; for obtaining at least one health benefit related to amelioration of the conditions associated with aging or age-related decline in NAD levels.

In one preferred embodiment, the present invention provides synergistic herbal compositions comprising; (i) a Punica granatum fruit rind extract containing calcium complex or chelate or metal salt of at least one phytochemical selected from punicalagins, punicalin, ellagic acid, and gallic acid or mixtures thereof; (ii) a Punica granatum seed extract; and (iii) at least one additional ingredient selected from the extract, fraction, phytochemical or mixtures thereof derived from Mangifera indica, Piper nigrum, Tagetes erecta, and Syzygium cumini; for obtaining at least one health benefit related to amelioration of the conditions associated with aging or age-related decline in NAD levels.

In one preferred embodiment, the present invention provides synergistic herbal compositions comprising; (i) a Punica granatum fruit peel extract containing potassium complex or chelate or metal salt of at least one phytochemical selected from punicalagins, punicalin, ellagic acid, and gallic acid or mixtures thereof; (ii) a Punica granatum seed extract; and (iii) at least one additional ingredient selected from the extract, fraction, phytochemical or mixtures thereof derived from Mangifera indica, Piper nigrum, Tagetes erecta, and Syzygium cumini; for obtaining at least one health benefit related to amelioration of the conditions associated with aging or age-related decline in NAD levels.

In another embodiment, the present invention provides synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; wherein the metal is selected from magnesium, calcium, and potassium; and (ii) at least one additional ingredient selected from the extract, fraction, phytochemical or mixtures thereof derived from Mangifera indica, Piper nigrum, Tagetes erecta, and Syzygium cumini.

In another embodiment, the present invention provides synergistic herbal compositions comprising; (i) a Punica granatum whole fruit extract containing metal complex or chelate or metal salt of at least one phytochemical selected from punicalagins, punicalin, ellagic acid, and gallic acid or mixtures thereof; wherein the metal is selected from magnesium, calcium, and potassium; and (ii) at least one additional ingredient selected from the extract, fraction, phytochemical or mixtures thereof derived from Mangifera indica, Piper nigrum, Tagetes erecta (marigold) and Syzygium cumini.

In another embodiment, the present invention provides synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; wherein the metal is selected from magnesium, calcium, and potassium; and (ii) at least one additional ingredient selected from the extract, fraction, phytochemical or mixtures thereof derived from Punica granatum seed.

In another embodiment, the present invention provides synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; wherein the metal is selected from magnesium, calcium, and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; wherein the punicalagins are in the range of 1.0-40%, punicalin is in the range of 0.5-5%, ellagic acid is in the range of 0.5-5% and gallic acid is in the range of 0.1-5%; and metal content is in the range of 0.1-5.0%.

In another embodiment, the present invention provides synergistic herbal compositions comprising; (i) a Punica granatum fruit rind extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; wherein the concentration of Punica granatum fruit peel extract containing metal complex in the composition varies in the range of 5%-80% by weight, Punica granatum seed extract in the composition varies in the range of 0%-50% by weight and Mangifera indica or Piper nigrum or Tagetes erecta or Syzygium cumini extract, fraction or phytochemical in the composition varies in the range of 5%-50% by weight.

In another embodiment, the present invention provides synergistic herbal compositions as described above; wherein the extract or fraction or phytochemicals derived from Punica granatum, Mangifera indica or Piper nigrum or Tagetes erecta or Syzygium cumini is obtained from at least one plant part selected from the group comprising leaves, stems, tender stems, tender twigs, aerial parts, whole fruit, fruit peel, fruit rind, seeds, flower heads, root, bark, hardwood, rhizome or whole plant or mixtures thereof.

In another embodiment, the present invention provides synergistic herbal compositions as disclosed above; wherein the extract, fraction, phytochemical or mixtures thereof; are produced using at least one solvent selected from C1-C5 alcohols selected from ethanol, methanol, n-propanol, isopropyl alcohol; ketones selected from acetone and methylisobutyl ketone, chlorinated solvents selected from methylene dichloride and chloroform; C1-C7 hydrocarbons such as but not limited to hexane, pentane; esters such as but not limited to ethyl acetate; and water and mixtures thereof.

In another embodiment, the present invention provides synergistic herbal compositions as described above; wherein the extract, fraction or mixtures thereof; in the composition are standardized to at least one phytochemical reference marker compound or pharmacologically active marker; wherein phytochemical marker compound or group of phytochemical compounds is/are in the concentration range of 0.01% to 50% by weight of the composition.

In another embodiment, the present invention provides synergistic herbal compositions comprising; (i) a Punica granatum fruit rind extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; and (iv) further containing at least one component selected from pharmaceutically or nutraceutically or dietically acceptable excipients, carriers, and diluents.

In another embodiment, the present invention provides a process for the preparation of compositions comprising; (i) a Punica granatum fruit rind extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta, and Syzygium cumini; wherein the process consists of the steps of;
(a) extracting dried Punica granatum fruit powder with a suitable solvent(s);
(b) treating the solution with a metal compound;
(c) filtering the solution;
(d) evaporating the solvent and drying the residue to obtain P. granatum fruit extract containing metal complex;
(e) optionally adding Punica granatum seed extract;
(f) blending with at least one extract derived from Mangifera indica, Tagetes erecta, Piper nigrum, and Syzygium cumini; in the presence of a suitable solvent; and optionally suitable excipients;
(g) drying the product to get the composition.

In another embodiment, the present invention provides methods of obtaining at least one health benefit related to amelioration of the conditions associated with aging or age-related decline in NAD levels, which are selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress; wherein the method comprises supplementing human with an effective dose of a composition comprising; (i) a Punica granatum fruit peel/rind extract containing metal complex or chelate or metal salt of at least one phytochemical selected from punicalagins, punicalin, ellagic acid and gallic acid or mixtures thereof; wherein the metal is selected from magnesium, calcium and potassium; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; and (v) further containing optionally at least one component selected from pharmaceutically or nutraceutically or dietically acceptable excipients, carriers, and diluents.

In another embodiment, the present invention provides the use of a synergistic herbal composition comprising; (i) a Punica granatum extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; and (v) further optionally at least one component selected from pharmaceutically or nutraceutically or dietically acceptable excipients, carriers, and diluents; for obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

In another embodiment of the invention, the composition as disclosed above is formulated into a dosage form selected form dry powder form, liquid form, beverage, food product, dietary supplement, or any suitable form such as a tablet, a capsule, a soft chewable tablet, or gummy bear.

In another embodiment of the invention, the composition as disclosed above is formulated into nutritional/dietary supplements that can be contemplated/made into the dosage form of healthy foods, or food for specified health uses such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

In another embodiment of the invention, the composition as disclosed above is formulated into a controlled-release tablet, using controlled-release polymer-based coatings by the techniques including nanotechnology, microencapsulation, colloidal carrier systems, and other drug delivery systems for obtaining the desired therapeutic benefit.

Those of ordinary skilled in the art will appreciate that changes could be made to the embodiments described above without departing from the broad inventive concept thereof. It is understood, therefore, that this invention is not limited to the particular embodiments or examples disclosed herein, but is intended to cover modifications within the objectives and scope of the present invention as defined in the specification. The examples are given solely for the purpose of illustration and are not to be construed as limitations of the present disclosure, as many variations thereof possible without departing from the spirit of the disclosure.

### Example 1: Punica granatum Fruit Rind Water Extract Containing Magnesium Complex of Punicalagins, Punicalin, Ellagic Acid, and Gallic Acid (PGRMg-1)

Punica granatum fruit rind powder (50 g) was added to water (300 mL) and stirred at ambient temperature for 2 h. The mixture was filtered, and the extraction process was repeated with water (2×200 mL). To the combined water extract, magnesium oxide (267 mg) was added and stirred at ambient temperature for 1 h. The mixture was filtered, and the filtrate was evaporated under reduced pressure to give the product as a brown color solid (24.5 g). The product was analyzed for punicalagins and other phytochemicals by the HPLC method of analysis and found that the product contains 9.38% of punicalagins, 2.13% of punicalin, 2.32% of ellagic acid and 0.50% of gallic acid. Magnesium was estimated by ICP-MS, and found that the product contains 0.76% of magnesium.

### Example 1A: Punica granatum Fruit Rind Water Regular Extract (PGR)

For comparison, P. granatum extract was prepared using the following procedure: P. granatum fruit rind powder (50 g) was added to water (300 mL) and stirred at ambient temperature for 2 h. The mixture was filtered, and the extraction process was repeated with water (2× 200 mL). The combined water extract was evaporated under reduced pressure to give the product as a dark brown color solid (20.0 g). The product was analyzed for punicalagins by the HPLC method of analysis, and found that the product contains 9.20% of punicalagins.

### Example 2: Punica granatum Fruit Rind 70% Aq. Ethanol Extract Containing Magnesium Complex of Punicalagins, Punicalin, Ellagic Acid, and Gallic Acid (PGRMg-2)

P. granatum fruit rind powder (50 g) was added to 70% aq. ethanol (300 mL) and stirred at ambient temperature for 2 h. The mixture was filtered through celite, and the extraction process was repeated with 70% aq. ethanol (2× 200 mL). To the combined 70% aq. ethanol extract magnesium oxide (501 mg) was added, and stirred at ambient temperature for 2 h. The mixture was filtered, and the filtrate was evaporated under reduced pressure to give the product as a pale brown color solid (22.5 g). The product was analyzed for punicalagins and other phytochemicals by the HPLC method of analysis and found that the product contains 14.36% of punicalagins, 2.03% of punicalin, 2.28% of ellagic acid, and 0.56% of gallic acid. Magnesium was estimated by ICP mass and found that the product contained 0.91% of magnesium.

### Example 3: Punica granatum Fruit Rind 70% Aq. Ethanol Extract Containing Calcium Complex of Punicalagins, Punicalin, Ellagic Acid, and Gallic Acid

The extract was prepared using the procedure as disclosed in Example 2, with calcium oxide instead of magnesium oxide to give the product as a pale brown color solid (21.0 g). The product was analyzed for punicalagins by the HPLC method of analysis, and found that the product contains 14.98% of punicalagins. Calcium was estimated by ICP mass, and found that the product contained 1.05% of calcium.

### Example 4: Punica granatum Fruit Rind 70% Aq. Ethanol Extract Containing Potassium Complex of Punicalagins, Punicalin, Ellagic Acid, and Gallic Acid

The extract was prepared as described in Example 2 except potassium carbonate instead of magnesium oxide to give the product as a pale brown color solid (22.0 g). The product was analyzed for punicalagins by the HPLC method of analysis, and found that the product contains 15.43% of punicalagins. Potassium was estimated by ICP mass, and found that the product contained 1.95% of potassium.

### Example 5: Punica Granatum Seed Extracts

(a) Ethyl acetate extract (PGS-1): P. granatum seeds powder (250 g) was added to ethyl acetate (1.5 L) and stirred at ambient temperature for 1 h. The mixture was filtered through celite, and the extraction process was repeated with ethyl acetate (2× 1.0 L). The combined ethyl acetate extract was evaporated under reduced pressure to give the product as yellow-colored oil (40.2 g). The product was analyzed for tripunicylglycerol or punicic acid by the HPLC method of analysis, and found that the product contains 79.56% of tripunicylglycerol.

(b) Water followed by 50% ethanol extract (PGS-2): The extract (11 g) was obtained from 50 g of raw material by adopting a similar procedure described above by using water extraction first followed by 50% ethanol extraction of the residue and then concentration and blending of the extracts to obtain P. granatum seed extract (PGS-2). Tripunicylglycerol or punicic acid by HPLC is 2.02%.

(c) Ethyl acetate, 50% ethanol followed by water extract (PGS-3): The extract (20 g) was obtained from 50 g of raw material by adopting a similar procedure described above for sequential extraction using ethyl acetate, 50% ethanol, followed by water as extraction solvents. All three extracts were combined to obtain the P. granatum seed extract (PGS-3), tripunicylglycerol or punicic acid by HPLC is 27.81%.

### Example 6: Punica granatum Whole Fruit Extract Containing Magnesium Complex of Punicalagins, Punicalin, Ellagic Acid, and Gallic Acid (PGMg)

P. granatum whole fruit (100 g) was crushed into a paste, and 200 mL of water was added. The mixture was heated in a pre-heated bath at 85-90° C. for 2 h. The mixture was filtered, and the extraction process was repeated with water (200 mL). To the combined water extract was added MgO (140 mg, 15.0 eq.), and the mixture was stirred at rt for 2 h. The mixture was filtered and was evaporated to 100 mL of volume. To the spent raw material was added 90% aq. ethanol (200 mL), and the mixture was heated at 80° C. for 2 h. The mixture was filtered, and the extraction process was repeated with 90% aq. ethanol (2× 100 mL). The combined 90% aq. EtOH extract was added to the concentrated water extract and was evaporated under reduced pressure to give the product as a brown color solid (21.3 g). The product was analyzed by the HPLC method of analysis and found to contain 3.94% and 1.71% of punicalagins and tripunicylglycerol or punicic acid, respectively. Magnesium was estimated by ICP mass, and found that the product contained 0.60% of magnesium.

### Example 7: Preparation of Mangifera indica Seed Extracts

(a) Water extract (MI-1): The dried M. indica seed kernel (100 g) was pulverized, and the powder raw material was added to water (400 mL) at rt. The mixture was stirred at ambient temperature for 2 h, and the extract was filtered through celite. The extraction process was repeated with water (2×300 mL) under similar conditions. The combined extracts were filtered and evaporated under reduced pressure to obtain extract concentrate, which was further subjected to vacuum drying to give the water extract of M. indica seed as a brown color powder (9.9 g). The product was analyzed by the HPLC method of analysis and found to contain 6.00% of pentagalloyl glucose.

(b) 50% aq ethanol extract (MI-2): The 50% aq ethanol extract (9.0 g) was obtained from 100 g raw material by adopting a similar procedure using 50% aq ethanol as extraction solvent. Pentagalloyl glucose by HPLC is 14.87%.

(c) 90% aq ethanol extract (MI-3): The 90% aqueous ethanol extract (10.4 g) was obtained from 100 g raw material by adopting a similar procedure using 90% aq ethanol as extraction solvent. Pentagalloyl glucose by HPLC is 19.55%.

(d) 50% ethanol followed by 90% ethanol (MI-4): The dried M. indica seed kernel (100 g) was pulverized, and the powder raw material was added to 50% aq ethanol (400 mL) at rt. The mixture was stirred at ambient temperature for 2 h, and the extract was filtered through celite. The extraction process was repeated with 50% aq ethanol (2× 300 mL) under similar conditions. The spent RM was then further extracted with 90% aq ethanol (3×300 mL). The combined extracts were filtered and evaporated under reduced pressure to obtain extract concentrate, which was further subjected to vacuum drying to give the 50% ethanol extract and 90% ethanol extract blend of M. indica seed as a brown color powder (17.2 g). Pentagalloyl glucose by HPLC is 15.90%.

### Example 8: Preparation of Piper nigrum Fruit Extracts

(a) Water extract (PN-1): The dried P. nigrum fruit (100 g) was pulverized, and the powder raw material was added to water (700 mL) at rt. The mixture was stirred at ambient temperature for 1 h, and the extract was filtered through celite. The extraction process was repeated with water (2× 500 mL) under similar conditions. The combined extracts were filtered and evaporated under reduced pressure to obtain extract concentrate, which was further subjected to vacuum drying to give the water extract of P. nigrum fruit as a brown color powder (8.5 g). The product was analyzed for piperine by the HPLC method of analysis, and found that the product contains 2.52% of piperine.

(b) 50% aq ethanol extract (PN-2): The 50% aq ethanol extract (12.5 g) was obtained from 100 g of raw material by adopting a similar procedure using 50% aq ethanol as an extraction solvent. Piperine by HPLC is 24.43%.

(c) Ethanol extract (PN-3): The ethanol extract (8.0 g) was obtained from 100 g of raw material by adopting a similar procedure using ethanol as an extraction solvent. Piperine by HPLC is 37.27%.

(d) 90% ethanol extract (PN-4): The dried P. nigrum fruit (100 g) was pulverized, and the powder raw material was added to 90% aq ethanol (600 mL) at rt. The mixture was stirred at 55-60° C. for 2 h, and the extract was filtered through celite. The extraction process was repeated with 90% aq ethanol (2× 250 mL) under similar conditions. The combined extracts were filtered and evaporated under reduced pressure to obtain extract concentrate, which was further subjected to drying to give the 90% ethanol extract of P. nigrum fruit as a brown color powder (10.0 g). Piperine by HPLC is 29.21%.

### Example 9: Preparation of Tagetes erecta Flower Extracts

(a) 80% aq. ethanol extract containing quercetagetin and quercetagetin-7-O-glucoside (T.E-1): Dried marigold flower petals powder (50 g) was pretreated with hexane (2×500 mL) at reflux temperature for 2 h. To the resulting spent raw material was added 80% aq. ethanol (650 mL) and the mixture was heated at 70-80° C. for 2 h. The mixture was filtered, and the extraction process was repeated twice with 80% aq. ethanol (2× 500 mL). The combined 80% aq. ethanol layer was evaporated under reduced pressure up to 100 mL of volume and was extracted with n-butanol (3× 100 mL) at rt. The combined n-butanol layer was washed with water (100 mL), and the n-butanol layer was evaporated under reduced pressure to give the product as a pale brown solid (6.5 g). The product was analyzed by the HPLC method of analysis and found to contain 17.2% and 11.1% of quercetagetin and quercetagetin-7-O-glucoside respectively.

(b) 90% aq. ethanol extract containing quercetagetin (T.E-2): Dried marigold flower petals powder (100 g) was pretreated with hexane (2× 1.0 L) at reflux temperature for 2 h followed by water (1 L) under maceration for 14 h. To the resulting spent raw material was added 90% aq. ethanol (800 mL), and the mixture was heated at 70-80° C. for 2 h. The mixture was filtered, and the extraction process was repeated twice with 90% aq. ethanol (2×600 mL). The combined 90% aq. ethanol layer was evaporated under reduced pressure to give the product as a brown color solid (12.0 g). The product was analyzed by the HPLC method of analysis and found to contain 49.5% of quercetagetin.

### Example 10: Preparation of Syzygium cumini fruit extracts

(e) Ethanol extract (SC-1): The dried Syzygium cumini fruit (100 g) was pulverized, and the powder raw material was added to ethanol (700 mL) at rt. The mixture was stirred at ambient temperature for 2 h, and the extract was filtered through celite. The extraction process was repeated with water (2× 500 mL) under similar conditions. The combined extracts were filtered and evaporated under reduced pressure to obtain extract concentrate, which was further subjected to vacuum drying to give the water extract of S. cumini fruit as a brown color thick paste (8.9 g).

(f) 90% aq ethanol extract (SC-2): The 90% aq ethanol extract (9.5 g) was obtained from 100 g of raw material by adopting a similar procedure using 90% aq ethanol as extraction solvent.

Example 11: Preparation of various compositions of Punica granatum fruit rind water extract containing punicalagins, punicalin, ellagic acid, and gallic acid and magnesium (PGRMg-1), Punica granatum seed extract (PGS-2), and Mangifera indica seed kernel extract (MI-4).

Comp-1: C-1 was prepared by combining PGRMg-1, PGS-2, and MI-4 in a ratio of 8:1:1.

Comp-2: C-2 was prepared by combining PGRMg-1, PGS-2, and MI-4 in a ratio of 6:2:2.

Comp-3: C-3 was prepared by combining PGRMg-1, PGS-2, and MI-4 in a ratio of 5:2:3.

Comp-4: C-4 was prepared by combining PGRMg-1, PGS-2, and MI-4 in a ratio of 4:3:3.

Comp-5: C-5 was prepared by combining PGRMg-1, PGS-2, and MI-4 in a ratio of 4:1:5.

Comp-6: C-6 was prepared by combining PGRMg-1, PGS-2, and MI-4 in a ratio of 3:5:2.

Composition for comparison (comp-2A): Comp-2A was prepared by combining Punica granatum fruit rind regular water extract (PGR), Punica granatum seed extract (PGS-2), and Mangifera indica seed kernel extract (MI-4) in the ratio of 6:2:2.

### Example 12: Preparation of Various Compositions of Punica granatum Fruit Rind Water Extract Containing Punicalagins, Punicalin, Ellagic Acid, and Gallic Acid and Magnesium (PGRMg-1), Punica granatum Seed Extract (PGS-2), and Piper Nigrum Fruit 90% Ethanol Extract (PN-4)

Comp-7: C-7 was prepared by combining PGRMg-1, PGS-2, and PN-4 in a ratio of 8:1:1.

Comp-8: C-8 was prepared by combining PGRMg-1, PGS-2, and PN-4 in a ratio of 6:2:2.

Comp-9: C-9 was prepared by combining PGRMg-1, PGS-2, and PN-4 in a ratio of 5:2:3.

Comp-10: C-10 was prepared by combining PGRMg-1, PGS-2, and PN-4 in a ratio of 4:3:3.

Comp-11: C-11 was prepared by combining PGRMg-1, PGS-2, and PN-4 in a ratio of 4:1:5.

Comp-12: C-12 was prepared by combining PGRMg-1, PGS-2, and PN-4 in a ratio of 3:5:2.

Composition for comparison (comp-8A): Comp-8A was prepared by combining Punica granatum fruit rind regular water extract (PGR), Punica granatum seed extract (PGS-2), and Piper nigrum fruit 90% ethanol extract (PN-4) in the ratio of 6:2:2.

Example 13: Preparation of various compositions of Punica granatum fruit rind 70% aq. ethanol extract containing punicalagins, punicalin, ellagic acid, and gallic acid and magnesium (PGRMg-2), Punica granatum seed extract (PGS-2), and Mangifera indica seed kernel extract (MI-4).

Comp-13: C-13 was prepared by combining PGRMg-2, PGS-2, and MI-4 in a ratio of 8:1:1.

Comp-14: C-14 was prepared by combining PGRMg-2, PGS-2, and MI-4 in a ratio of 6:2:2.

Comp-15: C-15 was prepared by combining PGRMg-2, PGS-2, and MI-4 in a ratio of 5:2:3.

Comp-16: C-16 was prepared by combining PGRMg-2, PGS-2, and MI-4 in a ratio of 4:3:3.

Comp-17: C-17 was prepared by combining PGRMg-2, PGS-2, and MI-4 in a ratio of 4:1:5.

Comp-18: C-18 was prepared by combining PGRMg-2, PGS-2, and MI-4 in a ratio of 3:5:2.

### Example 14: Preparation of Various Compositions of Punica granatum Fruit Rind 70% Aq. Ethanol Extract Containing Punicalagins, Punicalin, Ellagic Acid, and Gallic Acid and Magnesium (PGRMg-2), Punica granatum Seed Extract (PGS-2), and Piper nigrum Fruit 90% Ethanol Extract (PN-4)

Comp-19: C-19 was prepared by combining PGRMg-2, PGS-2, and PN-4 in a ratio of 8:1:1.

Comp-20: C-20 was prepared by combining PGRMg-2, PGS-2, and PN-4 in a ratio of 6:2:2.

Comp-21: C-21 was prepared by combining PGRMg-2, PGS-2, and PN-4 in a ratio of 5:2:3.

Comp-22: C-22 was prepared by combining PGRMg-2, PGS-2, and PN-4 in a ratio of 4:3:3.

Comp-23: C-23 was prepared by combining PGRMg-2, PGS-2, and PN-4 in a ratio of 4:1:5.

Comp-24: C-24 was prepared by combining PGRMg-2, PGS-2, and PN-4 in a ratio of 3:5:2.

### Example 15: Preparation of Various Compositions of Punica granatum Fruit Rind Water Extract Containing Punicalagins, Punicalin, Ellagic Acid, and Gallic Acid and Magnesium (PGRMg-1), and Tagetes erecta (Marigold) Flower 90% Ethanol Extract (TE-2)

Comp-25: C-25 was prepared by combining PGRMg-1 and TE-2 in a ratio of 3:1.

Comp-26: C-26 was prepared by combining PGRMg-1 and TE-2 in a ratio of 2:1.

Comp-27: C-27 was prepared by combining PGRMg-1 and TE-2 in a ratio of 1:1.

Comp-28: C-28 was prepared by combining PGRMg-1 and TE-2 in a ratio of 1:2.

Comp-29: C-29 was prepared by combining PGRMg-1 and TE-2 in a ratio of 1:3.

Composition for comparison (comp-25A): Comp-25A was prepared by combining Punica granatum fruit rind water regular extract (PGR), and Tagetes erecta (marigold) flower extract (TE-2) in the ratio of 3:1.

### Example 16: Preparation of Various Compositions of Punica granatum Fruit Rind Water Extract Containing Punicalagins, Punicalin, Ellagic Acid, and Gallic Acid and Magnesium (PGRMg-1) and Syzygium cumini Fruit 90% Ethanol Extract (SC-2)

Comp-30: C-30 was prepared by combining PGRMg-1 and SC-2 in a ratio of 3:1.

Comp-31: C-31 was prepared by combining PGRMg-1 and SC-2 in a ratio of 2:1.

Comp-32: C-32 was prepared by combining PGRMg-1 and SC-2 in a ratio of 1:1.

Comp-33: C-33 was prepared by combining PGRMg-1 and SC-2 in a ratio of 1:2.

Comp-34: C-34 was prepared by combining PGRMg-1 and SC-2 in a ratio of 1:3.

Composition for comparison (comp-32A): Comp-32A was prepared by combining Punica granatum fruit rind water regular extract (PGR), and Syzygium cumini fruit extract (SC-2) in a ratio of 1:1.

### Example 17: Preparation of Various Compositions of Punica granatum Fruit Rind 70% Aq. Ethanol Extract Containing Punicalagins, Punicalin, Ellagic Acid, and Gallic Acid and Magnesium (PGRMg-2), and Tagetes erecta Flower Extract (TE-2)

Comp-35: C-35 was prepared by combining PGRMg-2 and TE-2 in a ratio of 2:1.

Comp-36: C-36 was prepared by combining PGRMg-2 and TE-2 in a ratio of 1:1.

Comp-37: C-37 was prepared by combining PGRMg-2 and TE-2 in a ratio of 1:2.

Example 18: Preparation of various compositions of Punica granatum whole fruit extract containing punicalagins, punicalin, ellagic acid, and gallic acid and magnesium (PGMg); and Mangifera indica seed extract (MI-4).

Comp-38: C-38 was prepared by combining PGMg and MI-4 in a ratio of 2:1.

Comp-39: C-39 was prepared by combining PGMg and MI-4 in a ratio of 1:1.

Comp-40: C-40 was prepared by combining PGMg and MI-4 in a ratio of 1:2.

Example 19: Preparation of various compositions of Punica granatum fruit rind water extract containing punicalagins, punicalin, ellagic acid, and gallic acid and magnesium (PGRMg-1) and Punica granatum seed extract (PGS-2).

Comp-41: C-41 was prepared by combining PGRMg-1 and PGS-2 in a ratio of 2:1.

Comp-42: C-42 was prepared by combining PGRMg-1 and PGS-2 in a ratio of 1:1.

Comp-43: C-43 was prepared by combining PGRMg-1 and PGS-2 in a ratio of 1:2.

### Example 20: Formulation of the Compositions

Comp-44: A mixture of Punica granatum fruit rind water extract containing punicalagins, punicalin, ellagic acid, and gallic acid and magnesium (PGRMg-1, 540 g), Punica granatum seed extract (PGS-2, 180 g), Mangifera indica seed kernel extract (MI-4, 180 g), rice maltodextrin (80 g) and syloid (20 g) was blended in a double polybag. The product was pulverized through 0.5 mm mesh and sifted through #40 sieves to give the composition as a fine powder (Comp-44).

Comp-45: The mixture of Punica granatum fruit rind water extract containing punicalagins, punicalin, ellagic acid, and gallic acid and magnesium (PGRMg-1, 510 g) and Punica granatum seed extract (PGS-2, 170 g) was added to water (1000 mL) and stirred for a few min. To the above mixture, added Piper nigrum fruit extract (PN-4, 220 g) slowly and stirred for a few minutes, and further added microcrystalline cellulose (80 g) and stirred for a few minutes. The contents were dried at ambient temperature under reduced pressure to give the product as flakes. These flakes are homogenously blended with colloidal silicon dioxide (20 g) in a polyethylene cover or any suitable blender. The material was pulverized further and sifted through the #40 sieve to give the composition a fine powder (Comp-45).

Comp-46: The mixture of Punica granatum fruit rind water extract containing punicalagins, punicalin, ellagic acid, and gallic acid and magnesium (PGRMg-1, 450 g) and Syzygium cumini fruit extract (SC-2, 450 g) was added to water (1000 mL) and stirred for a few min. Then added rice maltodextrin (80 g) to the mixture and stirred for few min. The contents were dried at ambient temperature under reduced pressure to give the product as flakes. These flakes are homogenously blended with colloidal silicon dioxide (20 g) in a polyethylene cover or any suitable blender and pulverized the material and further sifted through #40 sieves to give the composition as a fine powder (Comp-46).

Comp-47: The mixture of Punica granatum fruit rind water extract containing punicalagins, punicalin, ellagic acid, and gallic acid and magnesium (PGRMg-1, 675 g) & Tagetes erecta flower extract (TE-2, 225 g) was added to 70% aqueous ethanol (900 mL) and stirred for few min. Then added water (1.8 L) followed by rice maltodextrin (80 g) to the mixture at ambient temperature and stirred for a few min. The contents were dried at ambient temperature under reduced pressure to give the product as flakes. These flakes are homogenously blended with colloidal silicon dioxide (20 g) in a polyethylene cover or any suitable blender and pulverized the material and further sifted through #40 sieves to give the composition as a fine powder (Comp-47).

### Example 21: Assay for Nicotinamide Adenine Dinucleotide (Oxidized, NAD) Boosting

Cell culture and Treatment: In a 48-well cell culture plate, an equal number of C2C12 mouse skeletal myoblasts (ATCC Cat #CRL-1772; Manassas, VA) were seeded. At 85-95% confluence, cells were induced to differentiate in DMEM medium (Himedia Cat #AL007S; Mumbai, India) supplemented with 2% Horse Serum (ATCC Cat #30-2040; Manassas, VA) for an additional 6 days to form myotubes. After differentiation, the C2C12 myotubes were treated with or without different concentrations of test samples or positive control (Resveratrol, Sigma Cat #R5010; St. Louis, MO) in MEM medium (Himedia Cat #AL047S; Mumbai, India) supplemented with 2% Horse serum for 48 hrs. C2C12 myotubes with 0.2% DMSO served as vehicle control. After the incubation, the medium was replaced with 50 μl of 1×PBS and 50 μl of base solution (0.2N NaOH) with 1% Cetyltrimethylammonium bromide (CTAB; Sigma Cat #H5882; St. Louis, MO) (Base treated sample). The plate was kept on the shaker briefly to ensure homogeneity and cell lysis. Protein was estimated using the Pierce BCA protein assay kit (Thermo Fisher Scientific Cat #23225; Waltham, MA). After normalization, 50 μg protein lysate was added to all the wells of a 96-well flat-bottom plate for further steps.

NAD Measurement: NAD measurement was performed using the Promega NAD/NADH-Glo™ kit (Cat #G9071; Madison, WI). Twenty-five microliters (containing 50 μg protein lysate) of each sample were added to empty wells in a 96-well flat-bottom plate for acid treatment. To these samples, 12.5 μl of 0.4N HCl was added (Acid treated sample). These wells contain acid-treated samples. The original sample wells are base-treated samples. The plate containing all samples was covered and incubated for 15 minutes at 60° C. After the incubation, the plate was equilibrated for 10 minutes at room temperature, and then 12.5 μl of 0.5M Trizma® base was added to each well of acid-treated cells to neutralize the acid. Twenty-five microliters of each sample were transferred into a 96-well, flat-bottom white plate (Corning Cat #3912; Corning, NY) for NAD measurement. Finally, 25 μl of NAD/NADH-Glo™ detection reagent was added and luminescence intensity was measured in a kinetic mode for upto 30 minutes. The percent increase of NAD over vehicle control as a result of sample treatment was calculated by the following formula:

% Increase in NAD over vehicle control=[(NAD concn. in sample-NAD concn. in vehicle control)]/NAD concn. in vehicle control×100.

The results are presented in tables: 4-12.

TABLE 4
Percent increase of NAD concentrations by the compositions
comprising Punica granatum fruit rind water extract
containing punicalagins, punicalin, ellagic acid, and gallic acid
and magnesium (PGRMg-1), Punica granatum seed extract
(PGS-2), and Mangifera indica seed extract (MI-4)

| | | | | | | | | | % increase of | |
|---|---|---|---|---|---|---|---|---|---|---|
| | PGRMg-1 | | PGS-2 | | MI-4 | | | Comp | NAD over control | |
| | Dose | % | Dose | % | Dose | % | | dose | Additive | |
| Comp # | μg/ml | increase | μg/ml | increase | μg/ml | increase | Ratio | μg/ml | (calculated) | Observed |
| C-1 | 0.8 | 22.79 | 0.1 | 2.29 | 0.1 | 2.61 | 8:1:1 | 1.0 | 27.69 | 36.87 |
| C-2 | 0.6 | 17.09 | 0.2 | 4.58 | 0.2 | 5.22 | 6:2:2 | 1.0 | 26.90 | 41.5 |
| C-3 | 0.5 | 14.25 | 0.2 | 4.58 | 0.3 | 7.84 | 5:2:3 | 1.0 | 26.66 | 34.59 |
| C-4 | 0.4 | 11.40 | 0.3 | 6.87 | 0.3 | 7.84 | 4:3:3 | 1.0 | 26.10 | 33.66 |
| C-5 | 0.4 | 11.40 | 0.1 | 2.29 | 0.5 | 13.06 | 4:1:5 | 1.0 | 26.75 | 34.77 |
| C-6 | 0.3 | 8.55 | 0.5 | 11.45 | 0.2 | 5.22 | 3:5:2 | 1.0 | 25.22 | 33.28 |

TABLE 5
Percent increase of NAD concentrations by the compositions comprising Punica granatum
fruit rind water extract containing punicalagins, punicalin, ellagic acid, and
gallic acid and magnesium (PGRMg-1), Punica granatum seed extract (PGS-2),
and Piper nigrum fruit 90% ethanol extract (PN-4)

| | | | | | | | | | % increase of | |
|---|---|---|---|---|---|---|---|---|---|---|
| | PGRMg-1 | | PGS-2 | | PN-4 | | | Comp | NAD over control | |
| | Dose | % | Dose | % | Dose | % | | dose | Additive | |
| Comp # | μg/ml | increase | μg/ml | increase | μg/ml | increase | Ratio | μg/ml | (calculated) | Observed |
| C-7 | 0.8 | 22.79 | 0.1 | 2.29 | 0.1 | 2.68 | 8:1:1 | 1.0 | 27.76 | 38.58 |
| C-8 | 0.6 | 17.09 | 0.2 | 4.58 | 0.2 | 5.36 | 6:2:2 | 1.0 | 27.03 | 38.96 |
| C-11 | 0.4 | 11.40 | 0.1 | 2.29 | 0.5 | 13.39 | 4:1:5 | 1.0 | 27.08 | 37.82 |
| C-12 | 0.3 | 8.55 | 0.5 | 11.45 | 0.2 | 5.36 | 3:5:2 | 1.0 | 25.35 | 35.14 |

TABLE 6
Percent increase of NAD concentrations by the compositions comprising Punica granatum fruit rind 70%
aq. ethanol extract containing punicalagins, punicalin, ellagic acid, and gallic acid and magnesium (PGRMg-2),
Punica granatum seed extract (PGS-2), and Mangifera indica seed kernel extract (MI-4).

| | | | | | | | | | % increase of | |
|---|---|---|---|---|---|---|---|---|---|---|
| | PGRMg-2 | | PGS-2 | | MI-4 | | | Comp | NAD over control | |
| | Dose | % | Dose | % | Dose | % | | dose | Additive | |
| Comp # | μg/mL | increase | μg/mL | increase | μg/mL | increase | Ratio | μg/ml | (calculated) | Observed |
| C-13 | 0.8 | 20.18 | 0.1 | 2.29 | 0.1 | 2.61 | 8:1:1 | 1.0 | 25.08 | 37.85 |
| C-14 | 0.6 | 15.14 | 0.2 | 4.58 | 0.2 | 5.22 | 6:2:2 | 1.0 | 24.94 | 35.42 |
| C-17 | 0.4 | 10.09 | 0.1 | 2.29 | 0.5 | 13.06 | 4:1:5 | 1.0 | 25.44 | 36.88 |
| C-18 | 0.3 | 7.57 | 0.5 | 11.45 | 0.2 | 5.22 | 3:5:2 | 1.0 | 24.24 | 35.69 |

TABLE 7
Percent increase of NAD concentrations by the compositions comprising Punica granatum fruit rind 70%
aq. ethanol extract containing punicalagins, punicalin, ellagic acid, and gallic acid and magnesium (PGRMg-2),
Punica granatum seed extract (PGS-2), and Piper nigrum fruit 90% ethanol extract (PN-4)

| | | | | | | | | | % increase of | |
|---|---|---|---|---|---|---|---|---|---|---|
| | | | | | | | | | NAD over | |
| | PGRMg-2 | | PGS-2 | | PN-4 | | | Comp | control | |
| Comp | Dose | % | Dose | % | Dose | % | | dose | Additive | |
| # | μg/mL | increase | μg/mL | increase | μg/mL | increase | Ratio | μg/ml | (calculated) | Observed |
| C-19 | 0.8 | 20.18 | 0.1 | 2.29 | 0.1 | 2.68 | 8:1:1 | 1.0 | 25.15 | 36.46 |
| C-20 | 0.6 | 15.14 | 0.2 | 4.58 | 0.2 | 5.36 | 6:2:2 | 1.0 | 25.07 | 35.34 |
| C-23 | 0.4 | 10.09 | 0.1 | 2.29 | 0.5 | 13.39 | 4:1:5 | 1.0 | 25.77 | 37.65 |
| C-24 | 0.3 | 7.57 | 0.5 | 11.45 | 0.2 | 5.36 | 3:5:2 | 1.0 | 24.37 | 36.14 |

TABLE 8
Percent increase of NAD concentrations by the compositions comprising
Punica granatum fruit rind water extract containing punicalagins,
punicalin, ellagic acid, and gallic acid and magnesium (PGRMg-1)
and Tagetes erecta flower extract (TE-2)

| | | | | | | | % increase of NAD | |
|---|---|---|---|---|---|---|---|---|
| | PGRMg-1 | | TE-2 | | | Comp | over control | |
| Comp | Dose | % | Dose | % | | dose | Additive | |
| # | μg/ml | increase | μg/ml | increase | Ratio | μg/ml | (calculated) | Observed |
| C-25 | 0.75 | 21.37 | 0.25 | 6.52 | 3:1 | 1.0 | 27.89 | 44.56 |
| C-26 | 0.67 | 18.99 | 0.33 | 8.69 | 2:1 | 1.0 | 27.68 | 42.63 |
| C-28 | 0.33 | 9.50 | 0.67 | 17.38 | 1:2 | 1.0 | 26.88 | 37.91 |

TABLE 9
Percent increase of NAD concentrations by the compositions comprising
Punica granatum fruit rind water extract containing punicalagins,
punicalin, ellagic acid, and gallic acid and magnesium (PGRMg-1)
and Syzygium cumini fruit extract (SC-2)

| | | | | | | | % increase of NAD | |
|---|---|---|---|---|---|---|---|---|
| | PGRMg-1 | | SC-2 | | | Comp | over control | |
| Comp | Dose | % | Dose | % | | dose | Additive | |
| # | μg/ml | increase | μg/ml | increase | Ratio | μg/ml | (calculated) | Observed |
| C-31 | 0.67 | 18.99 | 0.33 | 8.52 | 2:1 | 1.0 | 27.51 | 38.82 |
| C-32 | 0.5 | 14.25 | 0.5 | 12.78 | 1:1 | 1.0 | 27.02 | 40.19 |
| C-33 | 0.33 | 9.50 | 0.67 | 17.03 | 1:2 | 1.0 | 26.53 | 36.88 |

TABLE 10
Percent increase of NAD concentrations by the compositions comprising
Punica granatum fruit rind 70% aq. ethanol extract containing
punicalagins, punicalin, ellagic acid, and gallic acid and magnesium
(PGRMg-2) and Tagetes erecta flower extract (TE-2).

| | | | | | | | % increase of NAD | |
|---|---|---|---|---|---|---|---|---|
| | PGRMg-2 | | TE-2 | | | Comp | over control | |
| Comp | Dose | % | Dose | % | | dose | Additive | |
| # | μg/ml | increase | μg/ml | increase | Ratio | μg/ml | (calculated) | Observed |
| C-35 | 0.67 | 16.82 | 0.33 | 8.69 | 2:1 | 1.0 | 25.51 | 34.47 |
| C-37 | 0.33 | 8.41 | 0.67 | 17.38 | 1:2 | 1.0 | 25.79 | 33.78 |

TABLE 11
Percent increase of NAD concentrations by the compositions comprising
Punica granatum whole fruit extract containing punicalagins,
punicalin, ellagic acid, and gallic acid and magnesium (PGMg) and
Mangifera indica seed kernel extract (MI-4).

| | | | | | | | % increase of NAD | |
|---|---|---|---|---|---|---|---|---|
| | PGMg | | MI-4 | | | Comp | over control | |
| Comp | Dose | % | Dose | % | | dose | Additive | |
| # | μg/ml | increase | μg/ml | increase | Ratio | μg/ml | (calculated) | Observed |
| C-38 | 0.67 | 14.52 | 0.33 | 8.71 | 2:1 | 1.0 | 23.23 | 32.06 |
| C-40 | 0.33 | 7.26 | 0.67 | 17.41 | 1:2 | 1.0 | 24.67 | 33.58 |

TABLE 12
Percent increase of NAD concentrations by the compositions comprising
Punica granatum fruit rind water extract containing punicalagins,
punicalin, ellagic acid, and gallic acid and magnesium (PGRMg-1)
and Punica granatum seed extract (PGS-2).

| | | | | | | | % increase of NAD | |
|---|---|---|---|---|---|---|---|---|
| | PGRMg-1 | | PGS-2 | | | Comp | over control | |
| Comp | Dose | % | Dose | % | | dose | Additive | |
| # | μg/ml | increase | μg/ml | increase | Ratio | μg/ml | (calculated) | Observed |
| C-41 | 0.67 | 18.99 | 0.33 | 7.63 | 2:1 | 1.0 | 26.62 | 37.18 |
| C-43 | 0.33 | 9.50 | 0.67 | 15.26 | 1:2 | 1.0 | 24.76 | 33.43 |

### Example 22: Assay for CD38 Inhibition

The L6 Rat skeletal myoblasts (ATCC Cat #CRL-1458; Manassas, VA) were maintained in 75 cm² cell culture flasks in DMEM medium (Himedia Cat #AL007S; Mumbai, India) supplemented with 10% FBS. Upon reaching 80% confluence, cells were washed with 1×PBS, trypsinized, and subjected to centrifugation at 250×g for 10 minutes. The resultant cell pellet was washed with 1×PBS and was dissolved in 0.25 M Sucrose (Sigma Cat #S0389; St. Louis, MO) buffer (containing 40 mM of Tris base, pH 7.4) and stored at −80° C. The following day, the cell lysate was thawed, 1% TritonX100 (Sigma Cat #T8787; St. Louis, MO) was added and sonicated for 3 minutes with 1 minute ON: OFF cycle and centrifuged at 13000 rpm for 10 minutes at 4° C. The resultant supernatant containing the CD38 enzyme was stored on ice until use or at −80° C. for future use. Protein concentration was estimated using the Pierce BCA protein assay kit (Thermo Fisher Scientific Cat #23225; Waltham, MA), and 25 μg/well protein was utilized for each reaction. In a 96-well Black round bottom polystyrene plate (Sigma Cat #CLS3792; St. Louis, MO), 50 μl enzyme (equivalent to 25 μg protein), 50 μl test samples or positive control (Quercetin, Sigma Cat #Q4951; St. Louis, MO) at different concentrations, and 100 μl (50 μM) of Nicotinamide 1, N6-ethenoadenine dinucleotide (ENAD, Sigma Cat #N2630; St. Louis, MO) were added. In parallel, enzyme control (only enzyme and ENAD) and buffer control wells (only buffer and ENAD) were also maintained. Relative Fluorescence Units (RFU) at endpoint was measured after 1 hour of incubation in the dark at Ex/Em: 290/400 nm in a Perkin Elmer EnSpire multimode plate reader (Waltham, MA). The % Inhibition of CD38 activity was calculated by the following formula:

% Inhibition of CD38 activity=[(Normalized RFU of Enzyme alone)−(Normalized RFU of Sample)]/Normalized RFU of Enzyme alone×100

The results are presented in tables: 13-21.

TABLE 13
Percent inhibition of CD38 activity by the compositions comprising Punica granatum fruit rind
water extract containing punicalagins, punicalin, ellagic acid, and gallic acid and magnesium (PGRMg-1),
Punica granatum seed extract (PGS-2), and Mangifera indica seed extract (MI-4).

| | | | | | | | | | % inhibition of | |
|---|---|---|---|---|---|---|---|---|---|---|
| | PGRMg-1 | | PGS-2 | | MI-4 | | | Comp | CD38 | |
| Comp | Dose | % | Dose | % | Dose | % | | dose | Additive | |
| # | μg/mL | increase | μg/mL | increase | μg/mL | increase | Ratio | μg/mL | (calculated) | Observed |
| C-1 | 8.0 | 25.75 | 1.0 | 2.62 | 1.0 | 3.01 | 8:1:1 | 10 | 31.38 | 44.21 |
| C-2 | 6.0 | 19.31 | 2.0 | 5.23 | 2.0 | 6.02 | 6:2:2 | 10 | 30.57 | 48.77 |
| C-3 | 5.0 | 16.10 | 2.0 | 5.23 | 3.0 | 9.04 | 5:2:3 | 10 | 30.36 | 43.78 |
| C-4 | 4.0 | 12.88 | 3.0 | 7.85 | 3.0 | 9.04 | 4:3:3 | 10 | 29.76 | 43.29 |
| C-5 | 4.0 | 12.88 | 1.0 | 2.62 | 5.0 | 15.06 | 4:1:5 | 10 | 30.55 | 44.01 |
| C-6 | 3.0 | 9.66 | 5.0 | 13.08 | 2.0 | 6.02 | 3:5:2 | 10 | 28.76 | 42.94 |

TABLE 14
Percent inhibition of CD38 activity by the compositions comprising Punica granatum fruit rind water
extract containing punicalagins, punicalin, ellagic acid, and gallic acid and magnesium (PGRMg-1),
Punica granatum seed extract (PGS-2), and Piper nigrum fruit 90% ethanol extract (PN-4)

| | | | | | | | | | % inhibition of | |
|---|---|---|---|---|---|---|---|---|---|---|
| | PGRMg-1 | | PGS-2 | | PN-4 | | | Comp | CD38 | |
| Comp | Dose | % | Dose | % | Dose | % | | dose | Additive | |
| # | μg/mL | increase | μg/mL | increase | μg/mL | increase | Ratio | μg/mL | (calculated) | Observed |
| C-7 | 8.0 | 25.75 | 1.0 | 2.62 | 1.0 | 2.90 | 8:1:1 | 10 | 31.27 | 43.27 |
| C-8 | 6.0 | 19.31 | 2.0 | 5.23 | 2.0 | 5.81 | 6:2:2 | 10 | 30.35 | 42.89 |
| C-11 | 4.0 | 12.88 | 1.0 | 2.62 | 5.0 | 14.52 | 4:1:5 | 10 | 30.01 | 43.41 |
| C-12 | 3.0 | 9.66 | 5.0 | 13.08 | 2.0 | 5.81 | 3:5:2 | 10 | 28.54 | 40.67 |

TABLE 15
Percent inhibition of CD38 activity by the compositions comprising Punica granatum fruit rind 70%
aq. ethanol extract containing punicalagins, punicalin, ellagic acid, and gallic acid and magnesium (PGRMg-2),
Punica granatum seed extract (PGS-2), and Mangifera indica seed kernel extract (MI-4).

| | | | | | | | | | % inhibition of | |
|---|---|---|---|---|---|---|---|---|---|---|
| | PGRMg-2 | | PGS-2 | | MI-4 | | | Comp | CD38 | |
| Comp | Dose | % | Dose | % | Dose | % | | dose | Additive | |
| # | μg/mL | increase | μg/mL | increase | μg/mL | increase | Ratio | μg/mL | (calculated) | Observed |
| C-23 | 8.0 | 18.08 | 1.0 | 2.62 | 1.0 | 3.01 | 8:1:1 | 10 | 23.71 | 32.19 |
| C-24 | 6.0 | 13.56 | 2.0 | 5.23 | 2.0 | 6.02 | 6:2:2 | 10 | 24.81 | 34.75 |
| C-27 | 4.0 | 9.04 | 1.0 | 2.62 | 5.0 | 15.06 | 4:1:5 | 10 | 26.72 | 34.53 |
| C-28 | 3.0 | 6.78 | 5.0 | 13.08 | 2.0 | 6.02 | 3:5:2 | 10 | 25.88 | 36.66 |

TABLE 16
Percent inhibition of CD38 activity by the compositions comprising Punica granatum fruit rind 70% aq.
ethanol extract containing punicalagins, punicalin, ellagic acid, and gallic acid and magnesium (PGRMg-2),
Punica granatum seed extract (PGS-2), and Piper nigrum fruit 90% ethanol extract (PN-4)

| | | | | | | | | | % inhibition of | |
|---|---|---|---|---|---|---|---|---|---|---|
| | PGRMg-2 | | PGS-2 | | PN-4 | | | Comp | CD38 | |
| Comp | Dose | % | Dose | % | Dose | % | | dose | Additive | |
| # | μg/mL | increase | μg/mL | increase | μg/mL | increase | Ratio | μg/mL | (calculated) | Observed |
| C-29 | 8.0 | 18.08 | 1.0 | 2.62 | 1.0 | 2.90 | 8:1:1 | 10 | 23.60 | 32.56 |
| C-30 | 6.0 | 13.56 | 2.0 | 5.23 | 2.0 | 5.81 | 6:2:2 | 10 | 24.60 | 35.98 |
| C-33 | 4.0 | 9.04 | 1.0 | 2.62 | 5.0 | 14.52 | 4:1:5 | 10 | 26.18 | 35.12 |
| C-34 | 3.0 | 6.78 | 5.0 | 13.08 | 2.0 | 5.81 | 3:5:2 | 10 | 25.66 | 35.43 |

TABLE 17
Percent inhibition of CD38 activity by the compositions comprising Punica granatum
fruit rind water extract containing punicalagins, punicalin, ellagic acid, and gallic
acid and magnesium (PGRMg-1) and Tagetes erecta flower extract (TE-2)

| | | | | | | | % inhibition of CD38 | |
|---|---|---|---|---|---|---|---|---|
| | PGRMg-1 | | TE-2 | | | Comp | activity | |
| Comp | Dose | % | Dose | % | | dose | Additive | |
| # | μg/ml | inhibition | μg/ml | inhibition | Ratio | μg/ml | (calculated) | Observed |
| C-13 | 7.5 | 24.14 | 2.5 | 7.68 | 3:1 | 10 | 31.82 | 53.45 |
| C-14 | 6.67 | 21.47 | 3.33 | 10.23 | 2:1 | 10 | 31.70 | 45.84 |
| C-16 | 3.33 | 10.72 | 6.67 | 20.48 | 1:2 | 10 | 31.20 | 44.37 |

TABLE 18
Percent inhibition of CD38 activity by the compositions comprising Punica granatum
fruit rind water extract containing punicalagins, punicalin, ellagic acid, and gallic
acid and magnesium (PGRMg-1) and Syzygium cumini fruit extract (SC-2)

| | | | | | | | % inhibition of CD38 | |
|---|---|---|---|---|---|---|---|---|
| | PGRMg-1 | | SC-2 | | | Comp | activity | |
| Comp | Dose | % | Dose | % | | dose | Additive | |
| # | μg/ml | inhibition | μg/ml | inhibition | Ratio | μg/ml | (calculated) | Observed |
| C-19 | 6.67 | 21.47 | 3.33 | 10.62 | 2:1 | 10 | 32.09 | 45.79 |
| C-20 | 5.00 | 16.10 | 5.00 | 15.94 | 1:1 | 10 | 32.04 | 46.13 |
| C-21 | 3.33 | 10.72 | 6.67 | 21.26 | 1:2 | 10 | 31.98 | 43.96 |

TABLE 19
Percent inhibition of CD38 activity by the compositions comprising Punica granatum
fruit rind 70% aq. ethanol extract containing punicalagins, punicalin, ellagic acid, and
gallic acid and magnesium (PGRMg-2) and Tagetes erecta flower extract (TE-2).

| | | | | | | | % inhibition of CD38 | |
|---|---|---|---|---|---|---|---|---|
| | PGRMg-2 | | TE-2 | | | Comp | activity | |
| Comp | Dose | % | Dose | % | | dose | Additive | |
| # | μg/ml | inhibition | μg/ml | inhibition | Ratio | μg/ml | (calculated) | Observed |
| C-35 | 6.67 | 15.07 | 3.33 | 10.23 | 2:1 | 10 | 25.30 | 36.29 |
| C-37 | 3.33 | 7.53 | 6.67 | 20.48 | 1:2 | 10 | 28.01 | 38.22 |

TABLE 20
Percent inhibition of CD38 activity by the compositions comprising Punica granatum
whole fruit extract containing punicalagins, punicalin, ellagic acid, and gallic acid
and magnesium (PGMg) and Mangifera indica seed kernel extract (MI-4).

| | | | | | | | % inhibition of CD38 | |
|---|---|---|---|---|---|---|---|---|
| | PGMg | | MI-4 | | | Comp | activity | |
| Comp | Dose | % | Dose | % | | dose | Additive | |
| # | μg/ml | inhibition | μg/ml | inhibition | Ratio | μg/ml | (calculated) | Observed |
| C-38 | 6.67 | 19.80 | 3.33 | 10.03 | 2:1 | 10 | 29.83 | 36.97 |
| C-40 | 3.33 | 9.89 | 6.67 | 20.09 | 1:2 | 10 | 29.98 | 37.17 |

TABLE 21
Percent inhibition of CD38 activity by the compositions comprising Punica granatum
fruit rind water extract containing punicalagins, punicalin, ellagic acid, and gallic
acid and magnesium (PGRMg-1) and Punica granatum seed extract (PGS-2).

| | | | | | | | % inhibition of CD38 | |
|---|---|---|---|---|---|---|---|---|
| | PGRMg-1 | | PGS-2 | | | Comp | activity | |
| Comp | Dose | % | Dose | % | | dose | Additive | |
| # | μg/ml | inhibition | μg/ml | inhibition | Ratio | μg/ml | (calculated) | Observed |
| C-41 | 6.67 | 21.47 | 3.33 | 8.71 | 2:1 | 10 | 30.18 | 39.53 |
| C-43 | 3.33 | 10.72 | 6.67 | 17.44 | 1:2 | 10 | 28.16 | 39.42 |

### Example 23: In-Vivo Study of NAD Levels Increase in Rats

The NAD boosting the efficacy of the selected individual extracts and their combinations were tested in aged male and female Wistar rats. Fifty-six aged (20 months old) and eight young (3 months old) Wistar rats were included in the study. On day 1 (0 hr), blood samples were collected (through an orbital plexus puncture) from each animal. These blood samples were evaluated for NAD concentrations using a standardized Liquid Chromatography-Mass Spectrometry (LC-MS/MS) method. The aged rats were randomized into seven groups (n=8); each group contained four male and four female rats. Each group received an oral gavage of either a vehicle (0.5% Carboxymethyl cellulose, CMC) or 250 mg/kg body weight of one of the test samples, i.e., PGRMg-1, PGS-2, MI-4, TE-2, Comp-44, Comp-47, for sixty consecutive days. For comparison, the young rats (n=8, four male and four female) were allocated to a separate group; they received the vehicle (0.5% CMC) through oral gavage during the study. At the end of the study period (i.e., on day 61), the blood samples were collected from the experimental animals; and blood cellular NAD concentrations were measured using the LC-MS/MS method.

TABLE 22
Blood NAD concentrations in herbal supplemented rats

| | Mean Blood NAD | | |
|---|---|---|---|
| | concentration (ng/ml) on | | % increase of NAD |
| Groups | Day 1 | Day 60 | level from Day 1 |
| Young control | 54379 | 55210 | 1.53 |
| Aged control | 36203 | 35878 | −0.90 |
| PGRMg-1 | 37289 | 43562 | 16.82 |
| PGS-2 | 38172 | 41882 | 9.72 |
| MI-4 | 36672 | 41752 | 13.85 |
| TE-2 | 38519 | 44407 | 15.29 |
| Comp-44 | 37793 | 48314 | 27.84 |
| Comp-47 | 38421 | 50344 | 31.03 |

1. A synergistic herbal composition comprising;
(i) a Punica granatum extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta (marigold) and Syzygium cumini; for obtaining at least one health benefit related to amelioration of the conditions associated with aging or age-related decline in NAD levels.

2. The synergistic herbal compositions as claimed in claim 1, wherein the metal is selected from magnesium, calcium, and potassium.

3. The synergistic herbal compositions as claimed in claim 1, wherein the Punica granatum extract is selected from Punica granatum fruit rind extract and Punica granatum whole fruit extract.

4. The synergistic herbal composition as claimed in claim 1, wherein the health benefit related to amelioration of the conditions associated with aging or age-related decline in NAD levels is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress.

5. The synergistic herbal composition as claimed in claim 1, wherein at least one phytochemical is selected from punicalagins, punicalin, ellagic acid, and gallic acid or mixtures thereof; wherein the punicalagins are in the range of 1.0-40%, the punicalin is in the range of 0.5-5%, the ellagic acid is in the range of 0.5-5% and the gallic acid is in the range of 0.1-5%; and the metal is selected from magnesium, calcium, and potassium; is present in the range of 0.1-5.0%.

6. The synergistic herbal composition as claimed in claim 1, wherein the concentration of Punica granatum extract contains metal complex varies in the range of 5%-80% by weight, Punica granatum seed extract in the composition varies in the range of 0%-50% by weight and the extract or fraction or phytochemical or mixtures thereof derived from Mangifera indica or Piper nigrum or Tagetes erecta or Syzygium cumini in the composition varies in the range of 5%-50% by weight.

7. The synergistic herbal composition as claimed in claim 1, wherein the extract or fraction is obtained from at least one plant part selected from the group comprising leaves, stems, tender stems, tender twigs, aerial parts, whole fruit, fruit peel, fruit rind, seeds, flower, flower heads, root, bark, hardwood, rhizome or whole plant or mixtures thereof.

8. The synergistic herbal composition as claimed in claim 1, wherein the extract, fraction, phytochemical or mixtures thereof; are produced using at least one solvent selected from C1-C5 alcohols, selected from ethanol, methanol, n-propanol, isopropyl alcohol; ketones selected from acetone and methylisobutyl ketone; chlorinated solvents selected from methylene dichloride and chloroform; C1-C7 hydrocarbons such as but not limited to hexane, pentane; esters such as but not limited to ethyl acetate; water and mixtures thereof.

9. The synergistic herbal composition as claimed in claim 1, wherein the extract, fraction or mixtures thereof; in the composition are standardized to at least one phytochemical reference marker compound or pharmacologically active marker; wherein phytochemical marker compound or group of phytochemical compounds is in the concentration range of 0.01% to 50% by weight of the extract.

10. The synergistic herbal compositions as claimed in claim 1, wherein the synergistic herbal composition further contain optionally at least one component selected from pharmaceutically or nutraceutically or dietically acceptable excipients, carriers, and diluents.

11. The synergistic herbal compositions as claimed in claim 10, wherein the pharmaceutically, nutraceutically or dietically acceptable excipients, carriers and diluents are selected from monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as starch and modified starch such as sodium starch glycolate, pre-gelatinized starch, soluble starch, ultrasperse A and other modified starches; Dextrins that are produced by hydrolysis of starch or glycogen such as yellow dextrin, white dextrin, maltodextrin, glucidex 12D, rice maltodextrin etc.; Polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide, syloids etc.; metallic stearates such as but not limited to calcium stearate, magnesium stearate, zinc stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; Natural pigments, flavors, Class I & Class II preservatives and aqueous, alcoholic, hydro-alcoholic, organic solutions of above listed ingredients alone or in combination.

12. The synergistic herbal compositions as claimed in claim 1, wherein the composition is formulated into a dosage form selected from dry powder form, liquid form, beverage, food product, dietary supplement, or any suitable form such as a tablet, a capsule, a soft gel capsule, a soft chewable tablet or gummy bear.

13. The synergistic herbal compositions as claimed in claim 1, wherein the composition is formulated into nutritional/dietary supplements that can be contemplated/made into the dosage form of healthy foods, or food for specified health uses such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

14. The synergistic herbal compositions as claimed in claim 1, wherein the composition is formulated into controlled-release tablets, using controlled release polymer-based coatings by the techniques including nanotechnology, microencapsulation, colloidal carrier systems, and other drug delivery systems for obtaining the desired therapeutic benefit.

15. A process for preparing synergistic herbal compositions comprising; (i) a Punica granatum extract containing metal complex or chelate or metal salt of at least one phytochemical selected from punicalagins, punicalin, ellagic acid, and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; wherein the process consists of the steps of;
(a) extracting dried P. granatum fruit powder with suitable solvent(s);
(b) treating the solution with a metal compound;
(c) filtering the solution;
(d) evaporating the solvent and drying the residue to obtain the P. granatum fruit extract containing metal complex;
(e) optionally adding Punica granatum seed extract;
(f) blending with at least one extract derived from Mangifera indica, Tagetes erecta, Piper nigrum, and Syzygium cumini; in the presence of a suitable solvent; optionally with a suitable excipient;
(g) drying the product to get the composition.

16. The process for preparing synergistic compositions as claimed in claim 15, wherein the suitable solvent is selected from but not limited to C1-C5 alcohols, like ethanol, methanol, n-butanol, water, and mixtures thereof.

17. The process for preparing synergistic compositions as claimed in claim 15, wherein the metal is selected from magnesium, calcium, and potassium.

18. The process for preparing synergistic compositions as claimed in claim 15, wherein the metal compound is selected from magnesium oxide, magnesium carbonate, magnesium hydroxide, calcium hydroxide, calcium carbonate, potassium hydroxide, and potassium carbonate.

19. A method of obtaining at least one health benefit related to amelioration of the conditions associated with aging or age-related decline in NAD levels, which is selected from but not limited to delaying aging process, promoting anti-aging activity, promoting longevity, promoting healthy aging, improving health span, extending healthy life, improving general health; delaying/preventing/treating neurodegenerative, cardiovascular, cataracts, hypertension and gastrointestinal diseases/conditions; improving glucose tolerance, muscle function, exercise capacity and cardiac function, neuro-behavioral functions including cognition and locomotor activity, endothelial function and mitochondrial function; and reducing oxidative stress; wherein the method comprises supplementing human with an effective dose of compositions comprising; (i) a Punica granatum extract containing metal complex or chelate or metal salt of at least one phytochemical selected from punicalagins, punicalin, ellagic acid and gallic acid or mixtures thereof; (ii) optionally containing a Punica granatum seed extract; and (iii) at least one additional ingredient selected from the extract, fraction, phytochemical or mixtures thereof derived from Mangifera indica, Piper nigrum, Tagetes erecta and Syzygium cumini; and (v) further containing optionally at least one component selected from pharmaceutically or nutraceutically or dietically acceptable excipients, carriers, and diluents.

20. (canceled)
